(19) United States
(12) Patent Application Publication
(10) Pub. No.: US 2006/0206118 A1
(43) Pub. Date: Sep. 14, 2006

(54) Percutaneous endoscopic access tools for the spinal epidural space and related methods of treatment

(76) Inventors: Daniel H. Kim, Los Altos, CA (US); Roy Chin, Pleasanton, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/362,431

(22) Filed: Feb. 23, 2006

Related U.S. Application Data
(63) Continuation-in-part of application No. 11/078,691, filed on Mar. 11, 2005.

(51) Int. Cl. A61B 17/88; A61F 2/44; A61F 2/46

(52) U.S. Cl. 606/86; 623/17.16

(57) ABSTRACT

Several alternative spinal access devices are described. A number of alternative methods for performing therapies in the spinal region using the described spinal access devices are also described.

### CROSS-REFERENCE TO RELATED APPLICATIONS

This application is a continuation-in-part of U.S. patent application Ser. No. 11/078,691 filed on Mar. 11, 2005, incorporated herein in its entirety.

### FIELD OF THE INVENTION

The present invention relates to methods and apparatuses for providing percutaneous access to portions of the spine, delivering devices and agents via the access as well as performing various therapeutic treatments to the spine and surrounding tissue. More particularly, devices and methods described herein may be used for example to perform annulus repair, herniated disc repair, denervation of neurological tissue, dispensing pharmacological agents and/or cell or tissue therapy agents, diagnosing disc degeneration and bony degeneration; nucleus decompression; as well as disc augmentation.

### BACKGROUND OF THE INVENTION

FIG. 1A is a posterior lateral view of two vertebral bodies 20 separated by an intervertebral disc 10. The intervertebral disc 10 is a cushion like pad with top and bottom endplates adjoining the bone surfaces of each adjacent vertebral body 20. From this posterior vantage point, access to the disc 10 is made difficult by the placement of the disc 10 relative to the vertebral structures such as, the spinous process 60, inferior facet joint 64, superior facet joint 66 and pedicle 67. FIG. 1B is a coronal view taken through a healthy disc 10 and the surrounding structures. The disc 10 has a nucleus pulposus 30. The nucleus pulposus 30 acts as a cushion for compressive stress. Around the nucleus pulposus 30 is an outer collar of a number of concentric fibrous rings called the annulus fibrosis 40. The annulus fibrosis 40 limits the expansion of the nucleus pulposus 30 when the spine is compressed. The rings of the annulus fibrosis 40 also bind the successive vertebrae 20 together, resist torsion of the spine, and assist the nucleus pulposus 30 in absorbing compressive forces. The annulus fibrosis 40 has an inner surface 41 adjacent the nucleus pulposus 30 and an outer surface 42 including annular nerve fibers 80. Also visible and a further challenge to accessing the disc 10 are a number of annular nerve fibers 80, spinal nerve roots 82, the epidural space 65, the dura 70, pia or spinal canal 72 and epidural venous plexus 81.

FIG. 1C shows an exemplary injury 50 to an intervertebral disc 10. In this illustration, the injury 50 is a herniated or prolapsed disc 52. This condition is commonly called a “slipped disc.” Severe or sudden trauma to the spine or nontraumatic pathology such as degenerative spine disease may cause a bulge, rupture, degeneration, or other area of injury 50 in one or more intervertebral discs. The annulus fibrosis 40 is thinnest posteriorly in the general direction of the spinous process 60, so the nucleus pulposus 30 usually herniates in that direction. The injury usually proceeds posterolaterally instead of directly posteriorly because the posterior longitudinal ligament strengthens the annulus fibrosis at the posterior sagittal midline of the annulus. The terms “posterior” and “posteriorly” mean the general posterior and posterolateral aspects of the disc 43 as distinguished from the anterior aspects of the disc (i.e., generally in the area of 41). As detailed above with respect to FIG. 1B, the posterior aspect of the annulus fibrosis 43 is also innervated by pain/sensory nerve fibers 80, ventral and/or dorsal nerve roots 82 and other delicate tissues including but not limited to the spinal dura 70. As such, a posterior injury of the nucleus pulposus 30 often impinges on one or more of these nerves. The resulting pressure on these nerves often leads to pain, weakness and/or numbness in the lower extremities, upper extremities, or neck region.

Additionally, once injured, the healing capacity of the annulus fibrosis is limited. Usually, healing occurs in the outer layers with the development of a thin fibrous film. However, the annulus fibrosis never returns to its original strength. In many cases, the annulus fibrosis never closes becoming highly susceptible to re-herniation or nucleus leakage.

Through degeneration or injury, the nucleus pulposus may dehydrate becoming less fluid and glutinous. The nucleus pulposis may bulge outward in all directions taking on a “roll” shape. This “roll” shape causes a reduction in mechanical stiffness of the joint which may result in joint instability. Over time, the disc may increasingly bulge until the “roll” extends beyond the normal circumference which compresses various nerves such as the neural foramen. A bulging nucleus pulposis results in pain, weakness, and numbness in the area of the body associated with the particular nerve.

Injured intervertebral discs are treated with bed rest, physical therapy, modified activities, and painkillers over time. There are a growing number of treatments that attempt to repair injured intervertebral discs repair thereby avoiding surgical removal of injured discs. Several treatments attempt to reduce discogenic pain. Many conventional treatment devices and techniques including open surgical approach with muscle dissection or percutaneous procedure without visualization are used to pierce a portion of the disc 10 under fluoroscopic guidance. For example, disc decompression is the removal or shrinking of the nucleus, thereby decompressing and decreasing the pressure on the annulus and nerves. Moreover, less invasive procedures, such as microlumbar discectomy and automated percutaneous lumbar discectomy remove the nucleus pulposis by suctioning through a needle laterally extended through the annulus. In addition, disc augmentation devices are implanted in order to treat, delay or prevent disc degeneration. Augmentation refers to both (1) annulus augmentation which includes repair of a herniated disc, support of a damaged annulus, closure of a torn annulus and (2) nucleus augmentation in which additional material is added to the nucleus. However, these devices provide little in the form of tactile sensation for the surgeon or allow the surgeon to atraumatically manipulate surrounding tissue. In general, these conventional systems rely on external visualization for the approach to the disc and thus lack any sort of real time, on-board visualization capabilities.

Furthermore, accurately diagnosing back pain is often more challenging than many people expect and often will involve a combination of thorough patient history and physical examination as well as a number of diagnostic tests. A major problem is the complexity of the various components of the spine as well as the broad range of physical symptoms experienced by individual patients. In addition, the epidural space contains various elements such as fats, connective tissue, lymphatics, arteries, veins, blood; and spinal nerve roots. These elements make it difficult to treat or diagnose within the epidural area because they collapse around any instrument or device inserted therein. For example, inserting an access device may result in damaging nerve roots or inserting a visualization device may result in blocked or reduced viewing capabilities. As such, the many elements within the epidural space limit the insertion, movement, and viewing capabilities of any access, visualization, diagnostic, or therapeutic device inserted into the epidural space.

The Prior art methods for diagnosing and treating an injured intervertebral disc do not provide real-time effective, minimally invasive, percutaneous capabilities. What is needed are minimally invasive techniques and systems that provide the capability to directly diagnose or repair the disc while minimizing damage to surrounding anatomical structures and tissues. Moreover, there is still a need for a method and device that allows a physician to effectively enter the epidural space of a patient, clear an area within the space to enhance visualization and use the visualization capability to diagnose and treat the disc injury.

### SUMMARY OF THE INVENTION

In one embodiment of the present invention there is provided a method of accessing a portion of the spine including percutaneously approaching a portion of the spine with an instrument having direct visualization capability; providing an access to a portion of the spine using the instrument; and delivering a device into the access provided using the instrument. In a further aspect, there is a method including delivering an expanding structure adjacent a portion of the spine to be accessed and expanding the expanding structure. In another aspect, the expanding structure is a mesh, a balloon or an expanding atraumatic element and may contain a material or marker to enhance visualization of the structure using an imaging modality outside of the body. In another aspect, the device is a monitor, a therapy delivery device, a stimulation device or a pharmacological therapy device or, alternatively, the device comprises an electrode, and wherein providing an access to a portion of the spine comprises providing an access to the spinal epidural space. In another aspect, the method includes implanting the device using the direct visualization capability of the instrument. In still another aspect, expanding the expanding structure comprises atraumatically deforming a portion of the spinal dura matter to create a sufficient working space. In still other aspects, a method includes providing an access to a portion of the spine, such as, providing an access to the spinal epidural space, the annulus, the layers of annulus, the disc nucleus. In still another aspect, the method also includes receiving visualization information from an imaging modality outside of the body such as, for example, from fluoroscopy, magnetic resonance imaging, and/or computer tomography. In still other aspects of the present method, the method includes using the direct visualization capability of the instrument to maneuver the instrument between a spinal nerve root and the spinal dura, to atraumatically manipulate the spinal nerve root and/or advancing the instrument while using a portion of the instrument to atraumatically manipulate the spinal nerve root. In yet another aspect of the present method, the method includes using the subject devices to deliver disc augmentation devices or nuclear augmentation devices. In another aspect of the present method the spinal access device may be used for diagnostic purposes.

In one embodiment of the present invention, there is provided a method for providing therapy to the spine by percutaneously introducing an instrument into a body; steering the instrument to a position adjacent the outer surface of the spinal dura matter using visualization information provided by the instrument; displacing the spinal dura matter with a portion of the instrument to enlarge the spinal epidural space; and advancing the instrument into the enlarged spinal epidural space. In a further aspect, the method may include placing the instrument in a position to provide therapy within the spinal region. In another aspect, the visualization information is provided from an image generated by a sensor located on the instrument. In another aspect, the sensor utilizes light to generate the image, the light has a wavelength between 1.5 to 15 microns, and/or the light has a wavelength suited to infrared endoscopy in the spinal region. In another aspect, the sensor utilizes acoustic energy to generate the image, the sensor utilizes an electrical characteristic to generate the image and/or the sensor distinguishes the type of tissue adjacent the sensor. In another aspect, displacing the spinal dura matter comprises displacing without piercing the spinal dura matter. In still another aspect, the method includes displacing the spinal dura matter with a portion of the instrument to enlarge the spinal epidural space is performed using an atraumatic tip of the instrument. In still another aspect, the method may include displacing the spinal dura matter with a portion of the instrument to enlarge the spinal epidural space by expanding a balloon or a structural member or an expandable cage to displace the spinal dura matter. In another aspect, the method also includes introducing a treatment device through a working channel in the instrument. In a further embodiment, the treatment device is a denervation device, a probe adapted to supply thermal energy to spinal tissue. In yet another embodiment, the treatment device is a disc augmentation device or a nuclear decompression device. In yet another embodiment, the treatment device is a stimulation electrode placed within the spinal column. In another embodiment, the subject devices are provided in a kit. In yet another embodiment, the treatment device is a placement of stimulation electrode at the appropriate location in the spinal column, with the aid of visualization. In another aspect, the method may be performed where in the step of percutaneously introducing is performed using a single incision. In another embodiment, the treatment device is a disc augmentation device or nucleus decompression device. In still a further aspect, the method includes using the instrument to dispense a compound to reduce, diminish or minimize epidural neural tissue scarring. In still another aspect, the method includes placing the instrument in a position to perform therapy on a posterior, exterior surface of the annulus, on spinal tissue adjacent the epidural space or by placing the instrument adjacent the annulus.

In still another aspect of the present invention, there is provided a spinal access device having an elongate body having a distal end and a proximate end, wherein the elongate body is adapted for percutaneous access to the spinal column; a direct visualization device on the elongate body distal end; a dissection tip on the elongate body distal end; and a working channel within the elongate body having an opening on the elongate body distal end. In another aspect, the dissection tip covers the direct visualization device and is transparent to the direct visualization device, and/or the dissection tip is self cleaning. In still another aspect, the direct visualization device is behind the dissection tip. In another aspect, the direct visualization device is wavelength based, the wavelength is in the visual spectrum, and/or the wavelength is transparent to blood. In another aspect, the visualization device uses acoustic energy or an electronic sensor. In yet another aspect, the dissection tip comprises an expandable structure such as a cage, balloon or a mesh in order to create a space for visualization by allowing clean saline to enter. In still another aspect, the distal end is steerable. In still further aspects, the diameter of the elongate body is less than about 5 millimeters, about 3 millimeters or about or less than 1 millimeter. In another aspect, the distal end is adapted for passage along a spinal epidural space to atraumatically deform spinal dura matter. In one aspect, the device includes another working channel adapted to dispense a pharmacological agent. In another aspect, the elongate body comprises a radio opaque marker or material. In another aspect, the device includes a sensor adapted to distinguish between different tissues and anatomical structures and the sensor may use a resistance, a capacitance, an impedance, an acoustic or an optical characteristic of tissue to distinguish between different tissues and anatomical structures. In another aspect, the device includes an annulus reinforcement element dimensioned for delivery via the working channel. In another aspect, the device include another working channel having an opening on the elongate body distal end, the another working channel opening is separate from the working channel opening or the another working channel joins the working channel opening. In another aspect, the elongate body further comprising a guide wire lumen. In still another aspect, the spinal access device is steerable. In another aspect, the spinal access device delivers a disc augmentation or nucleus decompression device. In yet another aspect of the invention, the spinal access device is used for diagnosing disc degeneration. In still another aspect of the invention, the subject devices are provided in a kit.

In another alternative embodiment of the present invention, there is provided a method for dispensing an active agent to a portion of the spine including percutaneously approaching a portion of the spine with an instrument having direct visualization capabilities; creating an access to a portion of the spine by maneuvering the instrument using the direct visualization capabilities of the instrument; and dispensing an active agent to a portion of the spine using the created access. In another aspect, creating the access comprises expanding a structure such as an expandable cage, a balloon or a mesh. In another aspect, the expanding step comprises atraumatically deforming the spinal dura matter. In another aspect, the instrument comprises a visual sensor having direct visualization capabilities. In another aspect, the instrument comprises an ultrasound sensor having direct visualization capabilities. In another aspect, the instrument comprises an electrical sensor having direct visualization capabilities. In another aspect, the instrument comprises a wavelength based sensor having direct visualization capabilities. In another aspect, the wavelength based sensor uses a wavelength in the visual spectrum. In another aspect, the wavelength based sensor uses a wavelength in the infrared spectrum. In another aspect, the wavelength based sensor uses a wavelength selected to see through blood. In another aspect, the wavelength based sensor uses a wavelength selected to visualize tissue. In another aspect, the tissue is neurological tissue. In another aspect, the method includes using visualization information from an imaging modality outside the body while percutaneously approaching a portion of the spine. In another aspect, the imaging modality outside the body comprises, fluoroscopy, magnetic resonance imaging or computer tomography. In another aspect, creating an access to a portion of the spine includes creating an access to the epidural space. In another aspect, creating an access to a portion of the spine includes creating an access to spinal neural tissue. In another aspect, creating an access to a portion of the spine comprises creating an access to one or more layers of the annulus. In another aspect, creating an access to a portion of the spine comprises creating an access to an outer surface of the disc annulus. In another aspect, creating an access to a portion of the spine comprises creating an access to the disc nucleus. In another aspect, creating an access to a portion of the spine is for delivery of a disc augmentation device or nuclear decompression device. In another aspect, the active agent is a drug to treat and/or prevent a disorder of the spine. In another aspect, the active agent comprises an anti-inflammatory agent, an analgesic agent, an anesthetic agent, an anti-cicatrizant agent, a wound healing agent or a lysis inducing agent. In another aspect, the method includes using a material or marker on the instrument to enhance visualization using an imaging modality outside the body. In another aspect, the material or marker on the instrument to enhance visualization comprises a radio opaque material or marker. In another aspect, the spinal access device is used to diagnose for bony degeneration. In still another aspect, a kit is provided which includes the subject devices.

In still another alternative embodiment of the present invention, there is provided an atraumatic spinal expansion device having an expandable structure having a distal end and a proximal end, the structure positionable between an expanded position and a stowed position, wherein, when in the expanded position, the structure is adapted to atraumatically deform spinal tissue. In one aspect, when in the expanded position the device forms a working channel within the device from the proximal end to the distal end. In another aspect, the device is adapted for percutaneous delivery to a portion of the spine while in the stowed position. In another aspect, the device is adapted to remain in place while a therapy is applied by a device disposed in the working channel. In another aspect, the structure comprises a balloon, a polymer, a memory metal frame, a drug coated structure or a structure comprising fibrous materials. In another aspect, the structure has a solid outer surface. In another aspect, the structure has a mesh outer surface. In another aspect, the device has a diameter of less than 5 mm in a stowed position. In another aspect, the device has a diameter of less than 3 mm in a stowed position. In another aspect, the device has a diameter of less than 1 mm in a stowed position.

In still another alternative embodiment of the present invention, there is provided a method of providing therapy to a portion of the spine including advancing a structure having a deployed position and a stowed position towards a spinal treatment site while the structure remains in the stowed position; and atraumatically deforming spinal tissue by changing the structure from the stowed position to a deployed position. In another aspect, the method includes creating a working area by changing the structure to the deployed position. In another aspect, the working area is within the structure in the deployed position. In another aspect, the working area is adjacent the structure in the deployed position. In another aspect, the includes advancing a therapeutic or diagnostic device to a position adjacent the atraumatically deformed spinal tissue. In another aspect, the method includes performing a therapeutic or diagnostic procedure with the therapeutic or diagnostic device while the structure is in the deployed position. In still additional aspects, the method includes repeatedly atraumatically deforming spinal tissue by changing the structure from the stowed position to the deployed position to provide a plurality of therapy positions. In another aspect, the plurality of therapy positions are positioned laterally on an annulus. In still another aspect, the method includes advancing the therapeutic or diagnostic device to at least one application position within each of the plurality of therapy positions. In another aspect, advancing a structure comprises percutaneously advancing a structure. In another aspect, the method includes advancing the structure to a therapy position and thereafter providing the therapeutic or diagnostic device to one or more application positions. In another aspect, the therapeutic or diagnostic device is an annulus reinforcement element. In another aspect, the annulus reinforcement element remains in place after the structure is removed. In another aspect, the device is a disc augmentation or nuclear decompression device.

In still another alternative embodiment, there is provided a method of providing a therapy to a portion of the spine including positioning a guide wire adjacent a portion of the spine; and advancing along the guide wire an instrument adapted to provide a therapy to a portion the spine. In another aspect, advancing along the guide wire comprises passing the guide wire through a working channel of the instrument. In another aspect, the method includes providing from a first lumen in the instrument a shield adapted to protect surrounding tissue from the therapy. In another aspect, the method includes providing from a second lumen in the instrument a therapy device adapted to provide a therapy to a portion the spine. In another aspect, the portion of the spine is the annulus. In another aspect, the instrument is adapted to apply energy to a portion of the spine.

In yet another alternative embodiment of the present invention, there is provided a method of performing a procedure in the spine including positioning a guide wire to form a pathway to a position adjacent a portion of the spine; advancing an instrument along the guide wire while using a portion of the instrument to atraumatically displace tissue adjacent the pathway to allow passage of the instrument; and atraumatically displacing the tissue adjacent the instrument using a device provided through a lumen in the instrument. In another aspect, atraumatically displacing tissue adjacent the instrument is performed by increasing the volume of the device. In another aspect, the method includes advancing a therapy device to a position adjacent a portion of the spine while atraumatically displacing the tissue adjacent the instrument using a device provided through a lumen in the instrument. In yet another aspect, the method also includes providing a therapy to a portion of the spine using the therapy device. In still another aspect, the therapy device is a disc augmentation device or a nuclear decompression device. In yet another aspect, the therapy device is an electrode stimulation device. In still another aspect, the therapy device is an ablation device. In another aspect, the method also includes diagnosing for disc degeneration.

In yet another alternative embodiment of the present invention, there is provided a method for providing therapy to the spine including introducing a spinal access device into a body; advancing the spinal access device through an opening formed by an interlaminar space within the spine; using a portion of the spinal access device to deform spinal dura; and advancing the spinal access device towards a posterior surface of an annulus. In another aspect, introducing the spinal access device comprises percutaneously introducing the spinal access device. In another aspect, using a portion of a spinal access device to deform spinal dura comprises atraumatically deforming the spinal dura. In another aspect, the method includes performing a therapy related to the annulus with a therapy device provided using the spinal access device. In another aspect, the opening formed by the interlaminar space and a posterior surface of the annulus are on the same spinal level. In another aspect, the method includes using an atraumatic deformation device provided via the spinal access device to deform the spinal dura. In another aspect, the method includes providing a therapy device adjacent the posterior annulus surface while using an atraumatic deformation device to deform the spinal dura.

In yet another alternative embodiment of the present invention, there is provided a device for providing therapy to the spine including a spinal access device comprising first and second working channels, a visualization port and an atraumatic tip; a shield delivery catheter dimensioned to be deliverable through the first working channel; a shield disposed on the shield delivery catheter; a therapy delivery catheter dimensioned to be deliverable through the second working channel; and a therapy device coupled to the therapy delivery catheter. In another aspect, the shield delivery catheter and the therapy device delivery catheter are joined together. In another aspect, the therapy device is biased to bend as it advances distal to the therapy device delivery catheter distal end. In another aspect, the therapy device is biased to bend into the same position regardless of the therapy position of the shield. In another aspect, the therapy device is biased to bend into a position dependant upon the therapy position of the shield. In another aspect, the shield is positionable between a stowed condition and a deployed condition. In another aspect, when the shield is in the stowed condition it is within the shield delivery catheter. In another aspect, when the shield is in the stowed condition it is disposed on the surface of the shield delivery catheter. In another aspect, the therapy device is movable relative to the shield. In another aspect, the therapy delivery catheter is movable relative to the shield delivery catheter. In another aspect, the device includes an extendable member disposed within and movable through the therapy delivery catheter and movable relative to the shield device delivery catheter. In another aspect, the therapy device delivery catheter and the shield delivery catheter have a preformed shape. In another aspect, the therapy device passes through the extendable member. In another aspect, the extendable member is positionable in one or more therapy positions relative to the shield. In another aspect, as the therapy device delivery catheter and the shield delivery catheter advance distal to the spinal access device the preformed shape positions the shield in different therapy positions. In another aspect, the therapy device is positionable into a plurality of application positions relative to each different therapy position.

Another alternative embodiment of the present invention provides a method for disc augmentation or nucleus decompression by introducing a spinal access device into a body; advancing the spinal access; using a portion of the spinal access device to deform spinal dura and create a working area; and advancing the spinal access device towards a treatment site. In another aspect, introducing the spinal access device comprises percutaneously introducing the spinal access device. In another aspect, using a portion of a spinal access device to deform spinal dura comprises atraumatically deforming the spinal dura thereby creating a working area.

In another alternative embodiment of the present invention, there is provided a system for disc augmentation or nucleus decompression including a spinal access device comprising a working channel, a delivery catheter; a visualization port and an atraumatic tip; and a disc augmentation or nucleus decompression device coupled to the delivery catheter. In another aspect, the present invention is used for diagnostic purposes. In another aspect of the invention, the system components are provided in a kit.

In another alternative embodiment of the present invention, there is provided a system for disc augmentation or nucleus decompression including a spinal access device comprising a working channel, a delivery catheter, a visualization port, aspiration ports, and an atraumatic tip wherein at least one disc augmentation or nucleus decompression device is coupled to the delivery catheter. In another aspect, the visualization port or the working channel and irrigation are performed together. In another aspect, the present invention is used for diagnostic purposes. In another aspect of the invention, the system components are provided in a kit.

In yet another alternative embodiment of the present invention there is provided a method of diagnosing disc degeneration within a patient. In an additional embodiment, the present invention diagnoses bony degeneration within a patient.

Another embodiment of the invention includes kits for use in practicing the subject methods.

### BRIEF DESCRIPTION OF THE DRAWINGS

FIG. 1A is a posterior lateral view of two vertebral bodies.

FIG. 1B is a coronal view of a healthy disc and surrounding spinal anatomy.

FIG. 1C is a coronal view of a herniated disc.

FIG. 2A is a perspective view of an embodiment of a spinal access device.

FIG. 2B is a perspective view of an alternative arrangement of elements in a spinal access device embodiment.

FIGS. 2C and 2D are perspective views of an embodiment of an atraumatic deformation device in a stowed condition (FIG. 2C) and a deployed condition (FIG. 2D).

FIG. 2E is a perspective view of an embodiment of a therapy device provided via the spinal access device of FIG. 2A.

FIG. 2F illustrates an atraumatic deformation device embodiment having two working channels.

FIGS. 2G, 2H and 2I illustrate side retracted, bottom and side deployed configurations, respectively, for a therapy device embodiment.

FIGS. 3A-7C illustrate various views of an embodiment of a method of performing a therapy in the spinal region using a posterior lateral approach.

FIGS. 8A-8C illustrate various views of an embodiment of a method for performing a therapy in the spinal region using an embodiment of the atraumatic manipulation device of FIG. 2F.

FIGS. 9A-14C illustrate various views of an embodiment of a method of performing a therapy in the spinal region using a lateral approach.

FIGS. 15A-20C illustrate various views of an embodiment of a method of performing a therapy in the spinal region using a posterior lateral approach to treat a torn annulus.

FIGS. 21A-21C illustrate an alternative spinal access device embodiment.

FIGS. 22A-24C illustrate embodiments of the spinal access device in use with a guide wire.

FIGS. 25A-25C illustrate a method of treating a portion of the spine with a spinal access device positioned in different application positions.

FIGS. 25D-25F illustrate an embodiment of the spinal access device of FIGS. 25A-25C with multiple therapy positions within a single application position.

FIGS. 25G-25I illustrate an aspect of a spinal delivery device embodiment having a movable therapy device delivery catheter.

FIGS. 25J-25L illustrate an aspect of a spinal delivery device embodiment having an extendable member within therapy delivery device catheter.

FIGS. 26A-26E illustrate a spinal access device embodiment in use with pre-formed delivery catheters.

FIGS. 26F and 26G illustrate aspects of pre-formed delivery devices of the present invention.

FIGS. 27A-30 illustrate various views of methods of performing a spinal therapy using a guide wire to position a spinal access device embodiment.

FIGS. 31-33 illustrate various annulus reinforcing element embodiments.

FIG. 34 is a perspective view of an embodiment of a spinal access device used to deliver an augmentation device or nucleus decompression device.

### DETAILED DESCRIPTION OF THE INVENTION

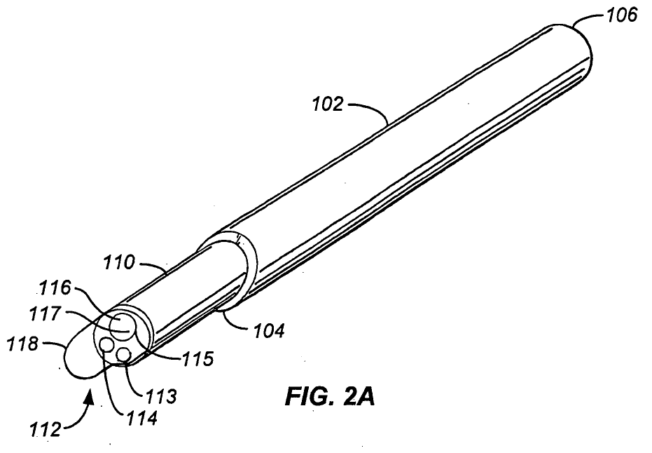

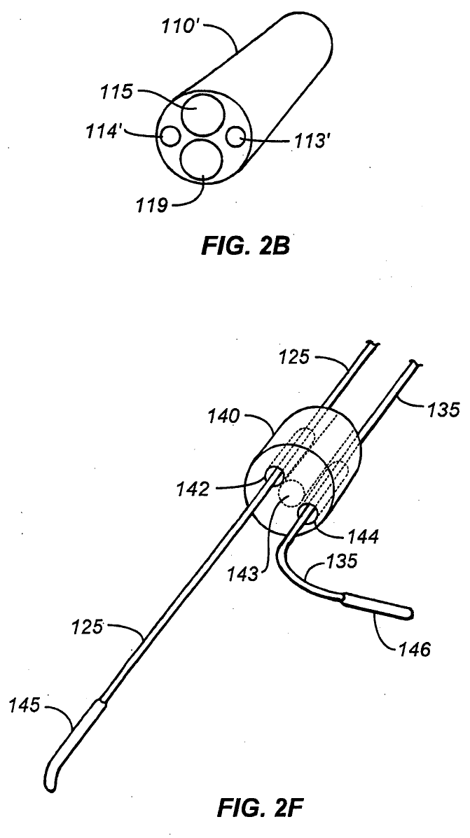

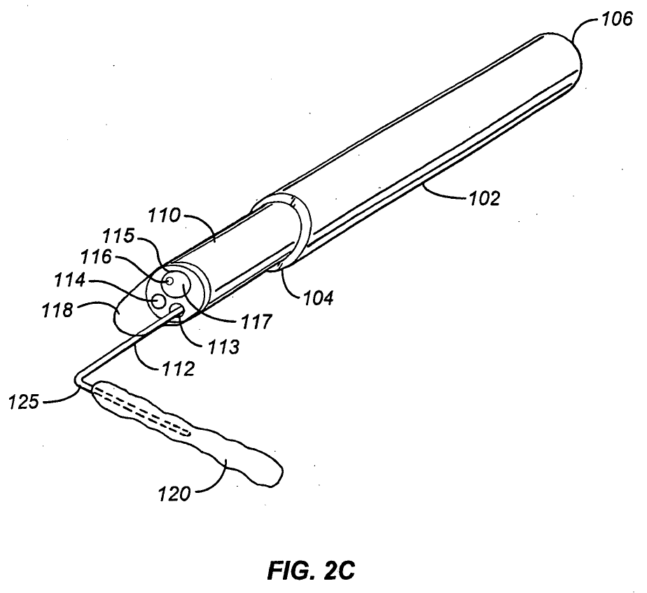

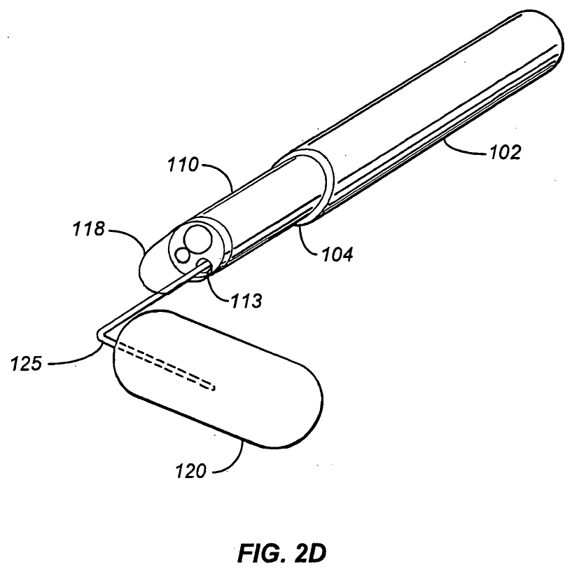

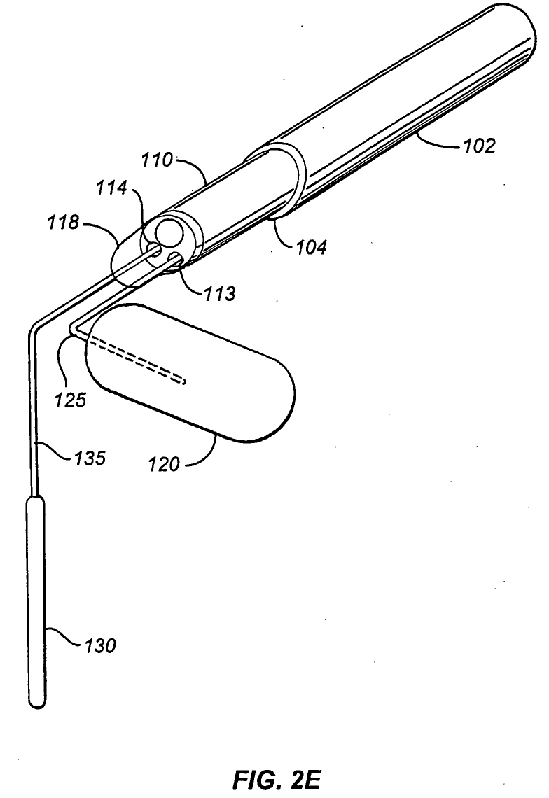

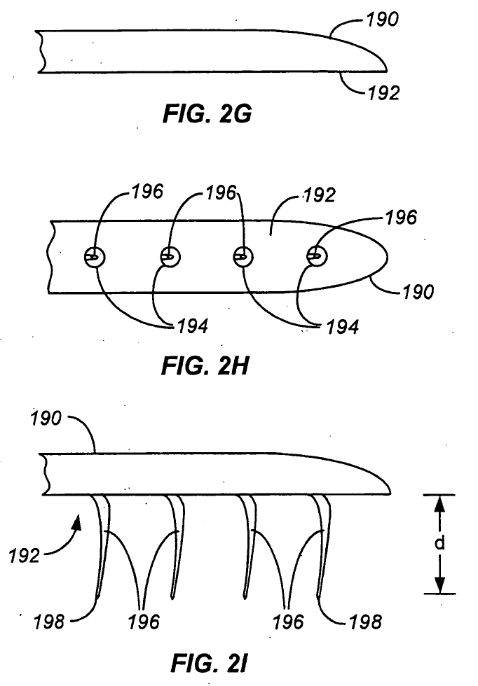

FIG. 2A illustrates an embodiment of a spinal access device 110 of the present invention. The spinal access device 110 includes a pair of working channels 113, 114 in a distal end 112. Additionally, the spinal access device 110 has a visualization port 115 covered by a shaped atraumatic tip 118. The visualization port 115 is used by illumination, visualization and/or imaging components to provide direct visualization capabilities for the spinal access device. In one aspect, the visualization port 115 may house one or more conventional illumination, visualization, analytical and/or imaging components used to illuminate, visualize, analyze or image the surrounding anatomical environment. The spinal access device 110 is illustrated within a conventional trocar or introducer 102. The trocar 102 has a distal tip 104, a proximal end 106 and a lumen 108 there through. The trocar 102 and lumen 108 are selected and sized to receive the spinal access device 110.

In one embodiment, the visualization port 115 includes within it an illumination port 116 and an imaging port 117 that are not visible in the view of FIG. 2A. In an alternative embodiment, rather than a single visualization port housing multiple components, each component may have a dedicated port. The visualization port 115, illumination port 116 and imaging port 117 provide access for conventional endoscopic imaging and/or medical imaging components as discussed above. In one aspect, either or both of the visualization port 115 and/or an illumination port 116 are forward looking. In another aspect (not shown), one or more ports are lateral looking. As described below, tissue differentiating sensors or their functional equivalent may also be provided through the ports.

One advantage of embodiments of the spinal access device of the invention is that steering the instrument may be performed using an image or information generated by a sensor located on the instrument. This image could come from a camera placed on the distal end of the device or be provided from a sensor or combination of sensors. In one aspect, the sensor utilizes light to generate the image. In another aspect the sensor is adapted to see through the bloody field as presented in the spinal region by selecting at least one infrared wavelength transparent to blood. In one embodiment, the at least one infrared wavelength transparent to blood presented in the spinal field has a wavelength between 1.5 to 15 microns. In one embodiment, the at least one infrared wavelength transparent to blood presented in the spinal field has a wavelength between 1.5 to 6 microns. In one embodiment, the at least one infrared wavelength transparent to blood presented in the spinal field has a wavelength between 6 to 15 microns. In yet another embodiment, the wavelength is selected or adapted for use in distinguishing nervous tissue from surrounding tissue and/or minimally vascularized nervous tissue. In yet another embodiment, the wavelength is selected to distinguish nervous tissue from muscle. Wavelength selection information and characterization and other details related to infrared endoscopy are found in U.S. Pat. No. 6,178,346; US Patent Application Publication US 2005/0014995 and US Patent Application Publication US 2005/0020914 each of which is incorporated by reference in its entirely for all purposes.

The visualization port 115 may contain, or the distal end of the device may include, a sensor used to generate images or identify tissue. In one example, the sensor utilizes acoustic energy to generate the image. In another example, the sensor utilizes an electrical characteristic to generate the image. In another example, the sensor distinguishes the type of tissue adjacent the sensor. Some properties used by the sensor to differentiate adjacent structures or tissue include resistance, capacitance, impedance, acoustic, optical characteristic of tissue adjacent the sensor or probe. Additionally, the sensor or image may be used to distinguish different types of tissue and identify neurological tissue, collagen or portions of the annulus. It is to be appreciated that the sensor could be a multi-sensor probe than can distinguish bone, muscle, nerve tissue, fat etc. to help locate the probe in the proper place.

The trocar 108 is guided using fluoroscopic or other external imaging modality to place the distal end 104 in proximity to a treatment area. In contrast to conventional procedures that attempt to fluoroscopically navigate a trocar tip around nerves and other tissue, the trocar 108 remains safely positioned away from sensitive structures and features. In one aspect, the trocar tip remains 5 cm or more from vulnerable nerve tissue. In another embodiment, the last 5 cm of travel to a therapy site is performed using direct visualization provided by an embodiment of the spinal access device.

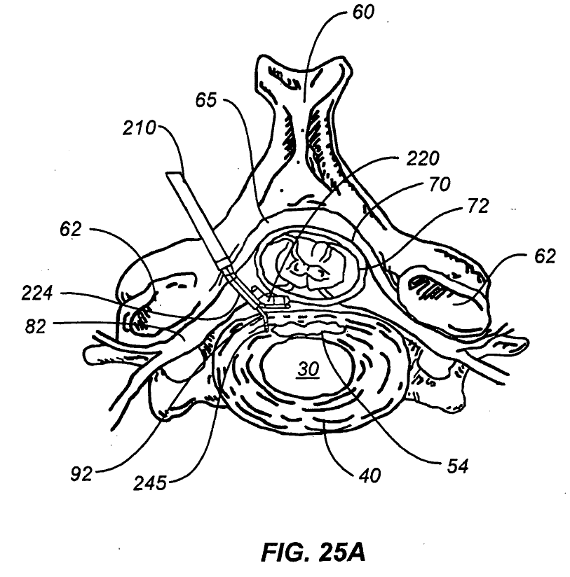

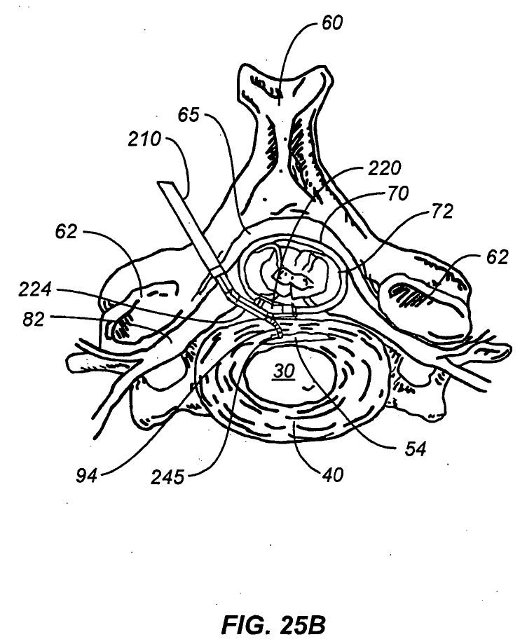

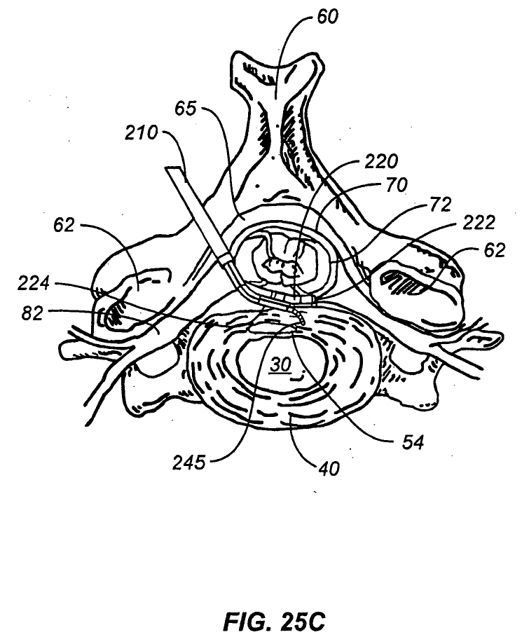

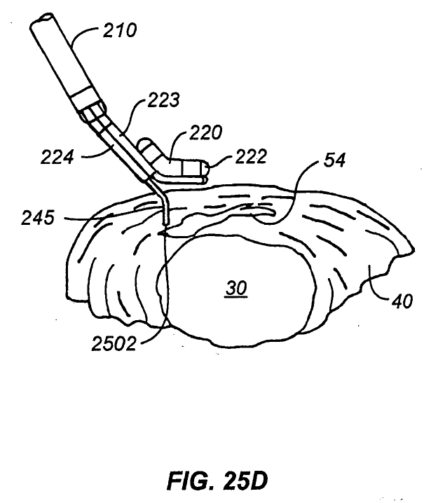

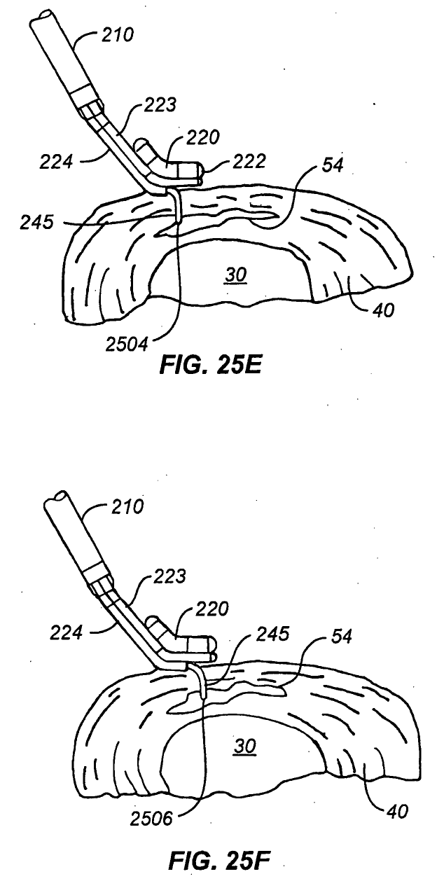

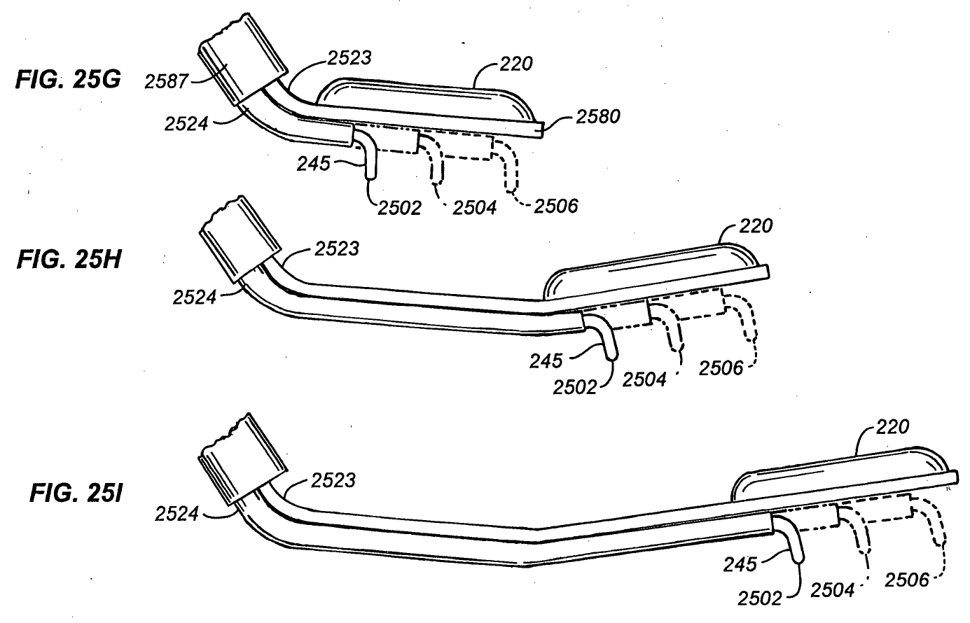

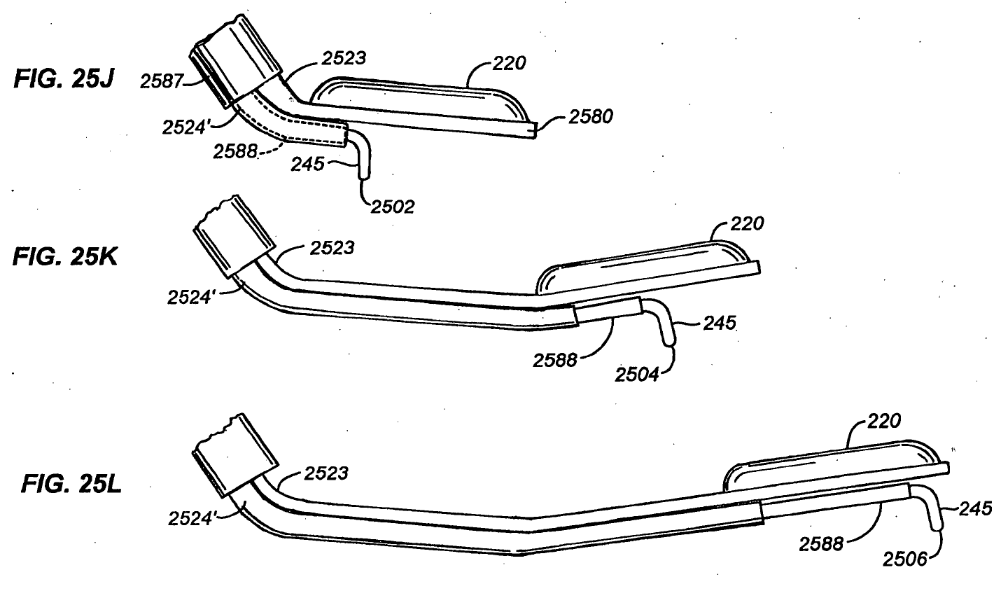

From the final trocar position, the spinal access device 110 then traverses the trocar and proceeds the remaining distance to the therapy or treatment site using the onboard visualization capabilities alone or in combination with the atraumatic tip 118 to identify, atraumatically displace and/or maneuver around nerves and other tissue as needed. In one aspect, the trocar distal end 104 is advanced within the body to a point where the steerable spinal access device 10 may then be used to manipulate surrounding tissue and structures to thereby traverse the remaining distance to one or more therapy or treatment sites (see, e.g., FIGS. 25A, 25B and 25C). In an alternative embodiment, the trocar 108 may house the spinal access device and thus use the direct visualization capabilities of the spinal access device to guide trocar positioning. In yet another alternative embodiment, both direct and external imaging are used to position the trocar distal end 104.

It is to be appreciated that embodiments of the spinal access device of the present invention provide a wide variety of steering configurations. In one aspect, embodiments of the spinal access device of the present invention are steerable in more than two axes. In one aspect, embodiments of the spinal access device of the present invention are steerable in two axes. In one aspect, embodiments of the spinal access device of the present invention are steerable in one axis. In one aspect, embodiments of the spinal access device of the present invention are non-steerable. In yet another alternative embodiment, the spinal access device is pre-formed into a shape that is adapted to access a portion of the spinal region.

In addition, the dimensions of the spinal access device embodiment used may be sized and selected based on the particular therapy being provided. For example, one embodiment of the spinal access device may be dimensioned for navigation about and the application of a therapy to the spinal region. In one aspect the spinal access device is sized to fit within the epidural space. In one embodiment the spinal access device 110 has a diameter of 5 mm or less. In one aspect, the one or more spinal access device working channels 113, 114 have a diameter of 4 mm. In another aspect, the one or more spinal access device working channels 113, 114 have a diameter of 3 mm. In still another aspect, the one or more spinal access device working channels 113, 114 have a diameter of 2 mm. In one additional aspect, the one or more spinal access device working channels 113, 114 have a diameter of 1 mm. In another aspect, the one or more spinal access device working channels 113, 114 have a diameter of less than 1 mm.

The atraumatic tip 118 is highly maneuverable as part of the steerable spinal access device 110 and provides a tactile sensation of the tissues and structures encountered. The atraumatic tip 118 is selected from a material that is transparent to the operation of the visualization port components. The tip 118 covers the distal end 112 about the visualization port 115 while leaving the working channels 113, 114 open for the introduction of instruments. In some embodiments, the atraumatic tip 118 is formed from rigid, clear plastic. In one embodiment, the atraumatic tip has a curved shape and no sharp edges, burrs or features that may pierce, tear or otherwise harm tissue that comes into contact with the atraumatic tip 118. Because of its design, the atraumatic tip 118 provides tactile feedback to the user of the rigidity, pliability or feel of the tissue or structures in contact with the tip 118. In one aspect, the atraumatic tip 118 also provides dissection capabilities along with the ability to displace surrounding tissue. The overall shape of the atraumatic tip allows nerves to be manipulated as the spinal access device is advanced without harming the nerve or causing pain (e.g., FIG. 10A). Moreover, because the tip is transparent to the visualization port 115, the user is also provided with a visual indication of the tissue adjacent the tip 118. The shape, surface contours and overall finish of the atraumatic tip 118 are selected to minimize impact when the tip comes into contact with structures, including nerves, muscle and the spinal dura among others. In one embodiment, the atraumatic tip 118 may be controllably inflatable. One use of such an embodiment could be that the tip 118 is contacted against tissue and then inflated to deform or move the tissue. In this manner, the tip may be inflated to create a working space in the surrounding tissue as well as provide a clearing for improved visibility. The spinal access device advanced into the working space. The tip inflated again to create another working space and so forth to advance the spinal access device in a spinal space. In addition, the access device may be used to provide saline or another type of cleaning solution to the working area for enhancing visualization. In another alternative embodiment, the distal tip 118 is moveable or articulated such that it may be used to nudge or prod surrounding tissue or structures. The nudge action is felt by the user and also provides a more tactile sense of tissue movement. The nudge may result from active movement of the tip under control of the user, movement caused by releasing the tip from a bias position or from other conventional techniques for manipulation of surgical implements.

FIG. 2B illustrates an arrangement components on the alternative embodiment spinal access device 110′. In the spinal access device 110′, the distal end 112 includes a visualization port 115, working channels 114′, 113′ having similar dimensions and a larger working channel 119. For clarity, the atraumatic tip 118 has been omitted but would be positioned over the distal end 112 to cover the visualization port 115 while leaving access to the working channels 113′, 114′ and 119. The size, number and arrangement of the working channels are readily adaptable depending upon the type of procedures performed. More or fewer working channels may be provided and the working channels need not have the same size and shape. In addition, the working channels may also be configured to perform auxiliary functions. In one specific example, a working channel is used to provide irrigation to assist in tissue dissection as the atraumatic tip is advanced in the spinal space. This irrigating working channel would be in communication proximally with a fluid source such as a syringe and distally with the distal end of the spinal access device so that the fluid exiting the irrigation working channel is directed to the distal portion of the spinal access device. In another specific example, the irrigation working channel or another working channel may be used to rinse the atraumatic tip or keep clear other portions of the spinal access tool.

FIGS. 2C, 2D and 2E illustrate how instruments may be introduced using the working channels 113, 114. In these illustrative embodiments, a first steerable catheter 125 is introduced through the working channel 113. An embodiment of an atraumatic manipulation device 120 is operably coupled to the distal end of the catheter 125. The atraumatic manipulation device 120 is used to temporarily deform or manipulate surrounding tissue, structure, or anatomical features. Embodiments of the atraumatic manipulation device 120 may also be used to assist with or perform therapy or treatment, shield surrounding tissue (e.g., FIG. 25A) or provide access for other devices (FIG. 2F). The manipulation device may be transferred to the surgical or treatment site in a compact or stowed condition (see, e.g., FIG. 2C) and then deployed according to the type of device used (see e.g., FIG. 2D).

It is to be appreciated that the atraumatic manipulation device 120 may manipulate surrounding tissue in a number of ways. First, by transitioning the device from a stowed to deployed configuration, the walls of the device will be urged outward against the surrounding tissue. Second, whether or not the device is deployed or stowed, the device 120 may be maneuvered using the catheter 125 to manipulate tissue. Third, atraumatic manipulation device 120 may cycled between the stowed and deployed configuration to assist in the advancement of the steerable spinal access device 110. As the device 120 expands, a work space or opening is created in the surrounding tissue thereby easing the advancement or atraumatic maneuverability of the spinal access device 110. Once the access device 110 is moved the manipulation device is returned to the stowed configuration and advanced toward a treatment location or other destination. Thereafter, the manipulation device 120 is deployed or otherwise used to deform surrounding tissue to make space available for the spinal access device 110 or other therapy or treatment device provided by working channel 114 (e.g., device 130 in FIG. 2E).

In the embodiment illustrated in FIGS. 2C and 2D, the atraumatic manipulation device 120 is an inflatable structure. The manipulation device 120 is adapted for delivery via the spinal access device. As such, in one embodiment, the manipulation device 120 is folded, compressed or stowed in such a manner that the manipulation device 120 is deliverable via an embodiment of the spinal access device. Additionally, the manipulation device 120 may be held by a sheath using techniques well known in the stent and stent delivery arts. Once the device 120 is positioned where desired, the sheath is removed to allow the device to transition to a deployed configuration.

Exemplary embodiments of the structure(s) 120 include balloons or other shaped inflatable structures used in angioplasty or other surgical procedures. Additionally, balloons used in intracranial procedures or other portions of the vasculature of comparable size to the spacing and/or working areas created in the spinal space using the present invention. There are a great many different shapes, sizes and functionality readily available in such balloons and many are well suited and easily adaptable for use in endoscopic spinal procedures. In one aspect, the balloon, when in a stowed configuration, is dimensioned to translate through a lumen or working channel in an embodiment of the spinal access toll described herein. The atraumatic manipulation device 120 may be shaped in virtually any shape desired to further spinal access. For example, the device 120 may be elongated, rounded, or other pre-formed shape. In one specific aspect, the device 120 has an elongate shape that follows the shape of an adjacent spinal structure. In one specific embodiment, the device 120 is adapted to follow a portion of the dura. In another specific embodiment, the device 120 is adapted to follow a portion of the annulus. In another aspect, the atraumatic manipulation device 120 includes a marker or other feature(s) making all or a portion of the device 120 perceptible using external imaging modalities. In one aspect, the marker or feature is a radio opaque marker.

Atraumatic device embodiments of the present invention are not limited to solid, inflatable embodiments. Non-solid structures such as mesh, scaffold structures, polymer stent-like structures, for example, may also be used to atraumatically deform spinal tissues. One example of a non-solid structure is a conventional coronary stent. Many of the delivery techniques used to deliver stents into the vasculature are applicable here for delivery into the spinal space to create greater and improved spinal access. The stent may also be a polymer stent or a stent with a coating to improve the atraumatic qualities of the stent to spinal tissues and structures. In another aspect, a suitable scaffold includes the collapsible scaffold structures used to deform and support tissue and maintain spacing between a radioactive source and the tissue being treated prior to and during brachytherapy.

In one embodiment, the surfaces of the atraumatic manipulation device 120 are expandable. For example, the atraumatic manipulation device 120 might be expandable using mechanical mechanisms, pneumatic mechanisms, or hydraulic mechanisms. In addition, the atraumatic manipulation device 120 may also contain sensing and/or monitoring devices such as a temperature thermo-couple. In an alternative embodiment, the atraumatic manipulation device 120 may include multiple layers and provide insulation or shielding to surrounding tissue by changing thermal and/or insulating properties either alone or in combination with expansion and contraction between the multiple layers. The change in properties could be accomplished by electrical, chemical, or mechanical properties of the layers, spaces between layers or through the use of a liquid, gas or other material inserted between layers or into a layer.

It is to be appreciated that while angioplasty and other balloon types may be suitable atraumatic manipulation devices, there are embodiments of the atraumatic manipulation device that are not circular in cross section or generally cylindrical as the balloons suited for use in the vasculature. In one aspect, the atraumatic manipulation device is adapted to conform to a portion of the spinal anatomy when in a deployed configuration. In another aspect, the atraumatic manipulation device is sized and adapted to conform to the shape of the annulus. In another specific aspect the atraumatic manipulation device has a preformed shape, a rounded shape, an elongated shape and combinations thereof. In one specific aspect the folded diameter of the atraumatic manipulation device is 10-40 thousandths of an inch. In another specific embodiment, the folded diameter of the atraumatic manipulation device is 25-35 thousandths of an inch. Other sizes are possible and may be selected based on the channel size of the spinal access device as well as the physical parameters of the patient's spinal area

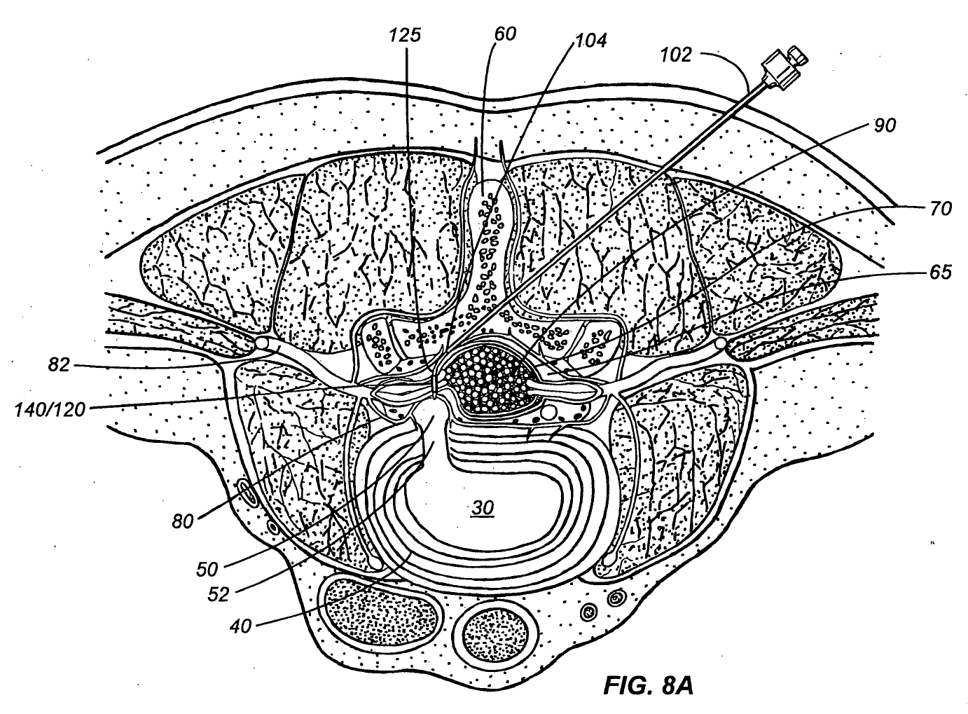

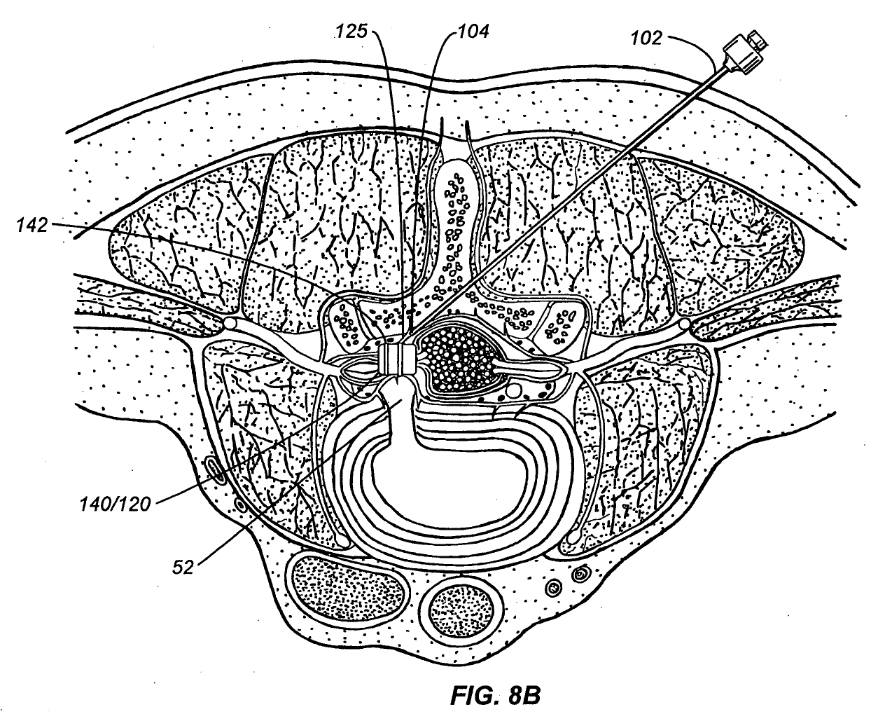

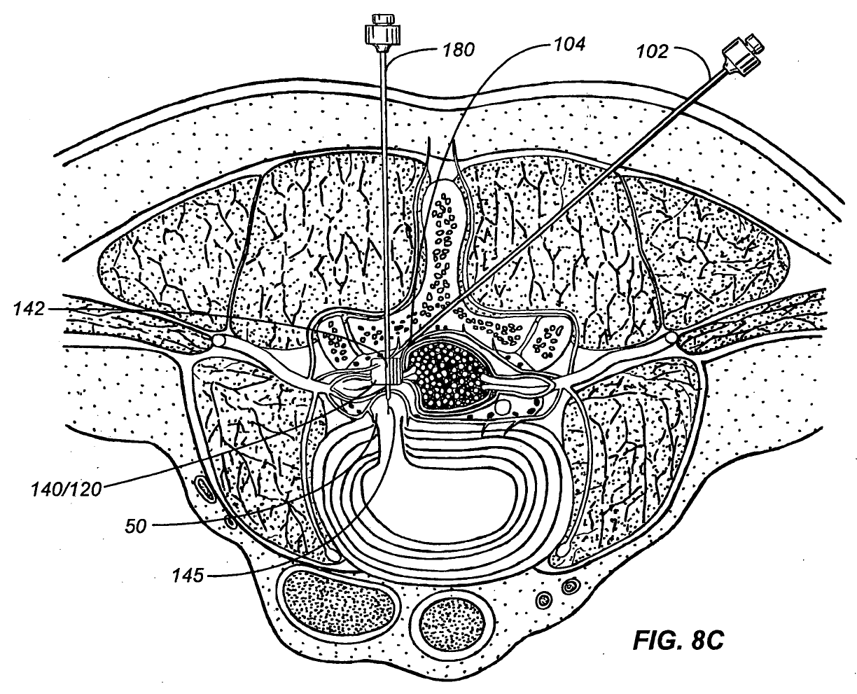

FIG. 2F illustrates an embodiment of an atraumatic manipulation device 140. The atraumatic manipulation device 140 not only provides the capabilities of the manipulation device 120 but also includes working channels to further assist in performing procedures. The manipulation device 140 is capable of both stowed and deployed configurations and is illustrated in a deployed configuration in FIG. 2F. Similar to the manipulation device 120, the manipulation device 140 is introduced in a stowed condition using a catheter via a working channel in an embodiment of the spinal device 110. Unlike the manipulation device 120, this embodiment of the manipulation device 140 provides two access lumens 142, 144. The access lumens 142, 144 run the length of the manipulation device 140 and are sized to allow passage of the catheters 125, 135 and instruments/devices 145, 146 respectively. One use of the manipulation device 140 is described below with regard to FIGS. 8A, 8B, and 8C. While two access lumens are illustrated, more or fewer may be provided in other than circular shapes and in a variety of different sizes depending upon use. For example, a third or central access lumen 143 (shown in phantom) may also be provided. A portion of the interior volume of device 140 may be filled with contrast solution in order to improve fluoroscopic visualization of the device 140.

FIG. 2E illustrates an embodiment of a therapy device 130 on a steerable catheter 135. The therapy device 130 is advanced through the working channel 114 and out into the treatment of working area created by the atraumatic manipulation device 120 alone or in combination with the atraumatic tip 118. The therapy device 130 may be any of a wide variety of devices suited to the type of therapy being performed. The therapy device 130 may configured and used to apply energy to surrounding tissue. The device 130 may also be a surgical instrument used to cut, pierce or remove tissue. Moreover, it is to be appreciated that the device 130 may be any conventional endoscopic instrument. The device 130 may include ultrasonic devices, motor driven devices, laser based devices, RF energy devices, thermal energy devices or other devices selected based on the spinal therapy being performed. For example, the device 130 may also be a mechanical device adapted to remove tissue such as a debrider or an aspirator.

In one aspect of the invention, the manipulation device 120 remains in place while the therapy device 130 is in use. In another aspect, once the working or therapy area has been created or accessed using the manipulation device 120, the manipulation device 120 may be removed thereby allowing working channel 113 to be used for another instrument or therapy device or to provide support for a procedure. For example, in the case where the therapy device 130 is a mechanical debrider, a suitable tool introduced via the working channel 113 may be used to assist in removal of tissue from the debridement. In another alternative embodiment, the manipulation device 120 remains in a deployed state and is detached from the catheter 125. In this way, the manipulation device 120 remains in place to provide a working access while also freeing the working channel 113 and the catheter 125 for other tasks. In yet another example of the flexibility of the spinal access device 110, the working channels may be used to provide access for the delivery of pharmacological agents to the access site either for application onto or injection into tissue.

The therapy device 130 and other therapy device embodiments described herein may be used to deliver energy to an intervertebral disc or portion thereof, or surrounding spinal tissue in support of a spinal therapy or treatment. The therapy device or energy applicator may be positioned on or within the structure being treated and may include more than one energy delivery device or energy applicator (e.g., FIG. 2I). Therapy devices or energy applicators may include one or more lasers fiber-optic strands, lenses, electrodes, wires, light bulbs, heating elements, and ultrasound transducers. A therapy device may have more than one energy-delivering side and each energy-delivering side may have more than one energy application region. A number of different types of energy may be utilized in the therapy device such as, for example, those described by Brett in U.S. patent application Ser. 10/613,678 filed Jul. 2, 2003 published as US 2004/0006379 and U.S. Pat. No. 6,673,063. Published Application US 2004/0006379 and U.S. Pat. No. 6,673,063 are incorporated herein by reference in their entirety and for all purposes.

The therapy device may be supplied with energy from a source external using a suitable transmission mode. For example, laser energy may be generated external to the body and then transmitted by optical fibers for delivery via an appropriate therapy device 130. Alternately, the therapy device may generate or convert energy at the therapy site, for example electric current from an external source carried to a resistive heating element within the therapy device. If energy is supplied to the therapy device, transmission of energy may be through any energy transmission means, such as wire, lumen, thermal conductor, or fiber-optic strand. Additionally, the therapy device may deliver electromagnetic energy, including but not limited to radio waves, microwaves, infrared light, visible light, and ultraviolet light. The electromagnetic energy may be in incoherent or laser form. The energy in laser form may be collimated or defocused. The energy delivered to a disc may also be electric current, ultrasound waves, or thermal energy from a heating element.

In addition, the therapy device may include multiple therapy delivery or energy application devices. Therapy device 190 illustrates an embodiment of a therapy device having multiple energy delivery devices 196 (FIGS. 2G-2I). The therapy device 190 is adapted for delivery using an embodiment of the spinal access device of the present invention and includes a treatment surface 192 containing a plurality of apertures 194. The apertures 194 are distributed across the treatment surface in any pattern useful for the therapy performed. In the illustrated embodiment, the pattern is a linear pattern. Visible within the apertures 194 and best seen in FIG. 2I are a plurality of energy delivery devices 196. The plurality of energy delivery devices 196 may be withdrawn into or extended—partially or fully—from the therapy device 190. In the illustrated embodiment, the energy delivery devices have a tapered shape and sharp distal end 198 configured to penetrate into tissue, such as the annulus or nucleus. Other configurations are possible.

In operation, the therapy device 190 is positioned so that the treatment surface 192 rests against the tissue to be treated using the energy delivery devices 196. For example, the treatment surface 192 could be placed against the posterior annulus so that the devices 196 extend a depth ‘d’ or portion of the depth ‘d’ into the tissue to denervate the annulus. The depth ‘d’ represents the maximum extension of the energy delivery devices 196 from the treatment surface 192. The depth ‘d’ will vary depending upon the specific energy delivery devices 196 used.

Alternatively, the devices 196 may extend a distance ‘d’ that allows the application of energy further into the annulus to treat, for example, a torn annulus. The shape and dimensions of the devices 196 may be altered depending upon the type of device used, energy or therapy provided.

Additionally, the therapy device may be a stimulation electrode device implanted within the spinal column. The subject device's direct visualization capabilities and the creation of a working space allow for the precise placement of the stimulation device for treating intervertebral degeneration.

In another aspect, there could be more than one therapy surface 192. In this aspect, the therapy device 190 itself may be used to penetrate into tissue to a desired location and then deploy one or multiple devices 196 from one or multiple therapy surfaces 192. It is to be appreciated that the therapy devices 196 may be devices used to apply energy into the tissue or may be adapted to deliver pharmacological agents or other compounds as described herein. Moreover, it is to be appreciated that embodiments of the spinal access devices described herein may also be used to dispense a compound, compounds or other pharmacological agents to reduce, diminish or minimize epidural neural tissue scarring.

First Exemplary Herniated Disc Treatment

As the illustrative treatment examples make clear, embodiments of the spinal access device and methods described herein are applicable to and enable novel surgical approaches to the spinal area. According to embodiments of the present invention, the spinal space may be approached using posterior mid-line, posterior lateral and/or far lateral approaches.

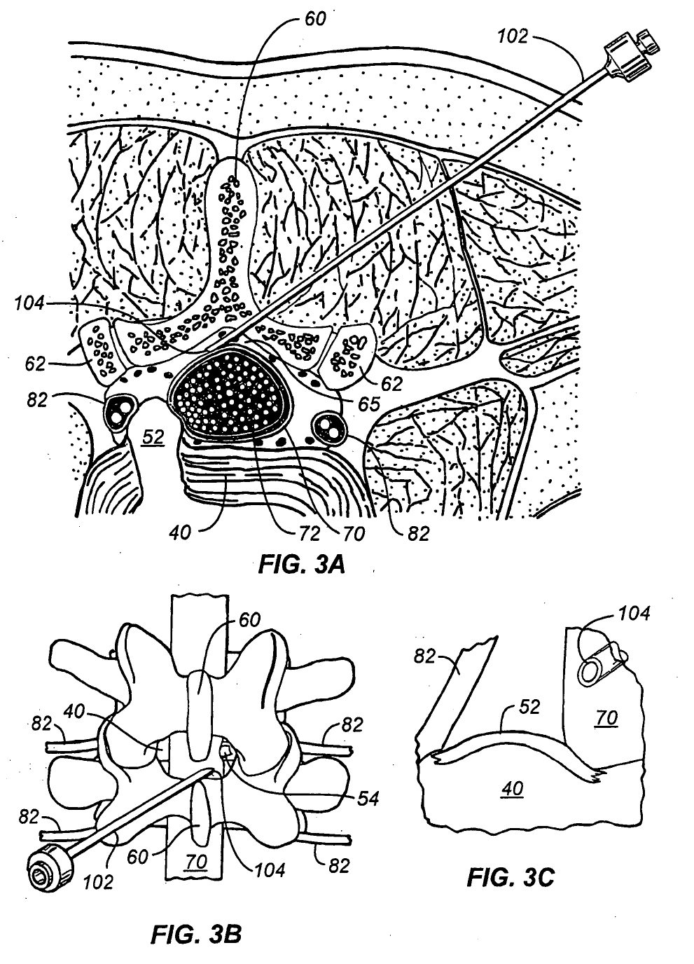

FIGS. 3A-7C illustrate an exemplary technique to shrink or remove a herniated disc 52. Each grouping of figures several views of a step of the procedure. The different views are: a coronal view (view A), a posterior view (view B) and an anterior view from the injury site (view C).

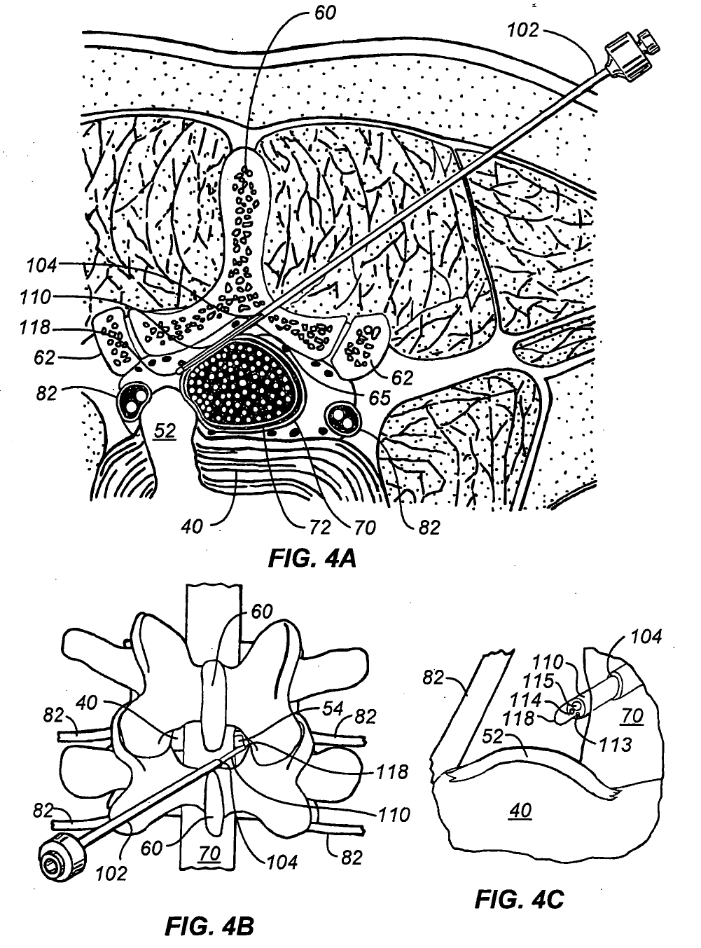

First, the trocar 102 or introducer is advanced using a conventional percutaneous approach to a position adjacent the injury or therapy site. In this illustrative embodiment, the trocar distal end 104 is positioned in the epidural space 65 using a posterolateral approach (FIG. 3A). The trocar distal end 104 is positioned between adjacent vertebra (FIG. 3B) towards the opening formed by the interlaminar space. As is clearly illustrated in FIG. 3A, the trocar distal tip 104 is advanced most of the distance towards the injury location, in this example a herniation 52. This step is a conventional step, and the trocar is introduced manually with guidance provided by an external imaging system such as fluoroscopy. However, unlike conventional spinal procedures, this marks the distal most movement of the trocar tip 104. As is made clear in the remaining steps and in the other examples, the trocar tip 104 remains a distance from the injury area. The remaining distance to the injury or therapy site is traversed using an embodiment of the spinal access device. In addition, the illustrative embodiment of FIGS. 4A, 4B and 4C show the trocar tip 104 in an area adjacent to but not in the epidural space 65. Numerous trocar positions are possible.

Next, the steerable spinal access device 110 is advanced through the trocar lumen and into the epidural space 65. The surgeon may use the tactile feedback from the atraumatic tip 118 to help guide the spinal access device 110. Advantageously, the atraumatic tip 118 is used to move the epidural fat and other tissue in the epidural space to aid in the advancement of the steerable spinal device 110 towards the treatment site. The distal end of the spinal device 110 as well as the atraumatic tip 118 are used to atraumatically deform the dura 70 as the spinal device 110 is advanced. The atraumatic tip 118 may also be configured to nudge tissue as discussed above. The surgeon may also be aided in guiding the spinal device 110 through use of direct visualization provided by the instruments in the visualization port 115. The spinal access device 110 is maneuvered into a position with a view of the treatment site (FIG. 4C). As such, the spinal access tool 110 is advanced towards the injury 54 using the visual or image data from the visualization port 115, tactile feedback from the atraumatic tip 118 and/or external image information. In one specific embodiment, the spinal access tool provides direct visualization of the spinal access area/approach to the interlaminar space, hence to the intraspinal epidural space to follow the lateral recess and reach the annulus posterior surface.

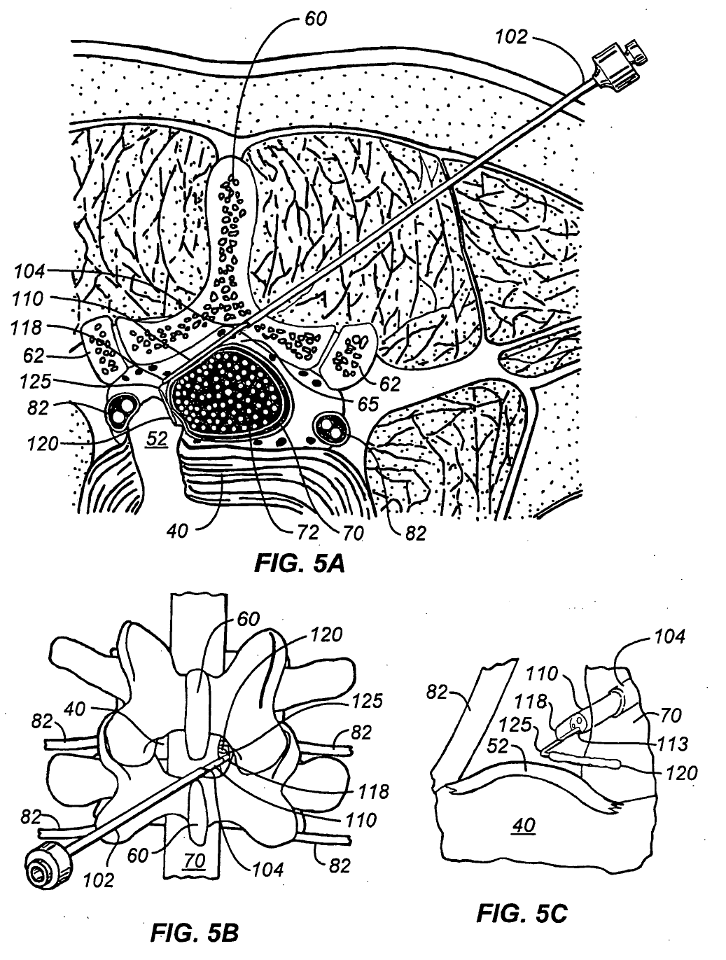

Next, an embodiment of the atraumatic manipulation device 120 is advanced through the working channel 113 using the steerable catheter 125 (FIGS. 5A, 5B and 5C). The atraumatic manipulation device 120 is shown in a stowed configuration. Even in the stowed configuration the manipulation device 120 may be used to move adjacent tissue, features and/or structures. For example, the manipulation device 120 may be used to deform the dura 70 (FIGS. 5A, 5C). The atraumatic manipulation device 120 is sized and shaped so that when deployed an appropriate working space or zone is created about the therapy site so that further treatment may be undertaken.

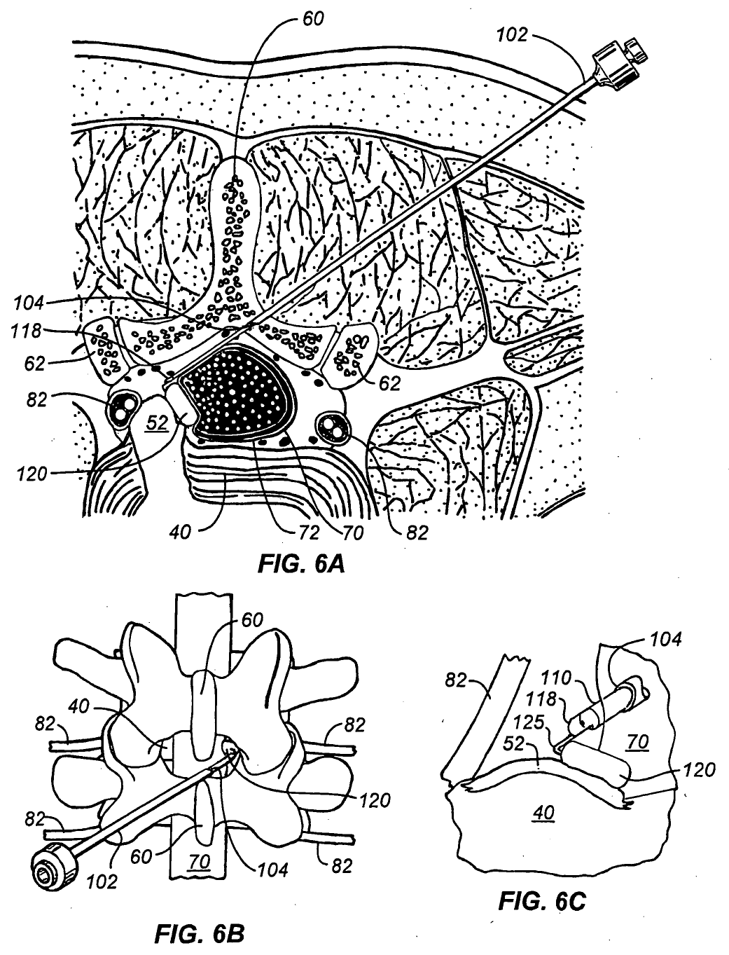

Next, after positioning the atraumatic manipulation device 120, the atraumatic manipulation device 120 is placed in a deployed configuration (FIGS. 6A, 6B, 6C). The dura 70 and surrounding tissue are further moved by the deployment action of the atraumatic manipulation device 120 to create a work space adjacent the therapy position, here the hernia 54. In alternative embodiments, the atraumatic manipulation device 120 may be partially deployed or cycled through deployed, stowed, and partially deployed positions to manipulate tissue and surrounding structures. As best seen in FIG. 6A, the spinal access device 110 and atraumatic manipulation device 120 are used to form an atraumatic spinal retractor.

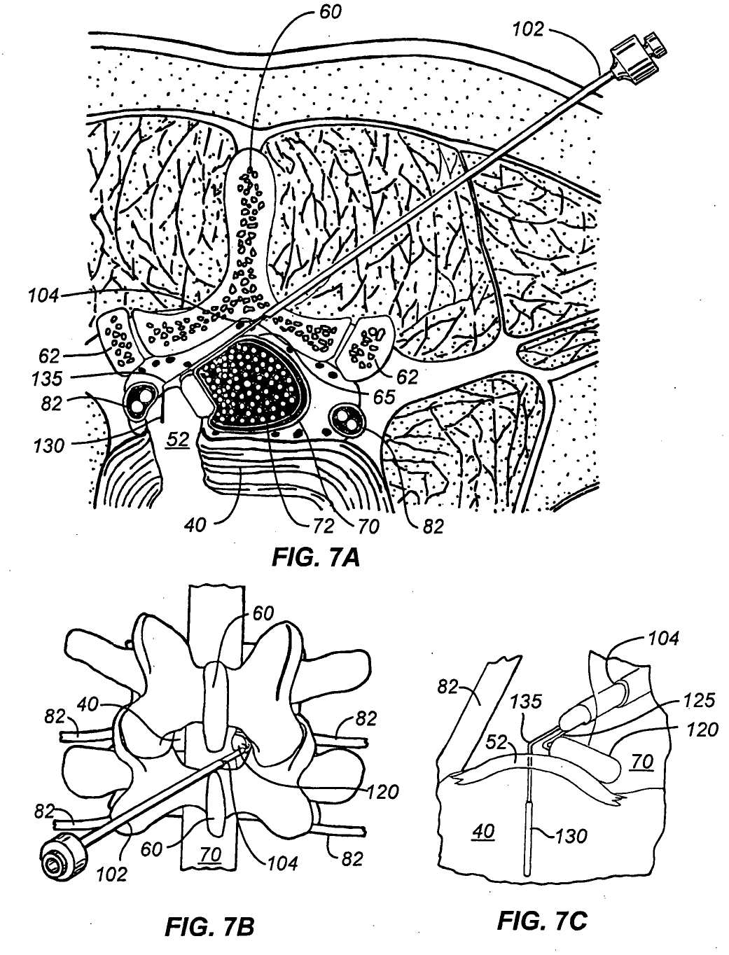

Next, a therapy device 130 attached to a catheter 135 is delivered to the therapy site via the working channel 114 (FIGS. 7A, 7B, 7C). The therapy device 130 is directed into the hernia 52 using the local, direct visualization capabilities of the spinal access device 110. In these illustrated embodiments, the device 130 penetrates into the hernia 52. It is to be appreciated that the device 130 may be inserted further into the nucleus 30 or withdrawn to treat the surface of hernia 52.

Second Exemplary Herniated Disc Treatment

FIGS. 8A, 8B and 8C illustrate an alternative two trocar treatment approach for a herniated disc treatment. First, a trocar 102 is positioned at or near the epidural space using a posterolateral approach as discussed above with regard to FIGS. 3A, 3B and 3C. Next, the steerable spinal access device 110 is maneuvered into a treatment position (FIG. 8A). Next, an embodiment of an atraumatic manipulation device 140 is maneuvered into position using a catheter and a working channel of the spinal access device 110. When the atraumatic manipulation device 140 is deployed, tissue is moved to create a treatment site and a working lumen 142 is provided (FIG. 8B). A second trocar 180 is then used in a mid-line approach toward a lumen of the device 140. An embodiment of a spinal access device is advanced through the second trocar 180 and then, using the direct visualization of the spinal access device, advanced towards and into a working lumen in the device 140. In the illustrated embodiment, the working lumen 142 is used. Using the pathway created by the second trocar 180 and the working lumen 142, a therapy device 145 is used at the injury site 50. In one embodiment, a single spinal access device may be used with the first trocar 100 to place the device 140. Next, the spinal access device is removed and used with the second trocar 180 to visualize and access a working lumen in the device 140. In an alternative embodiment, the atraumatic manipulation device 140 may include fluoroscopic contrast material (solid or liquid) to aid in guiding the spinal access device.

First Exemplary Torn Annulus Treatment

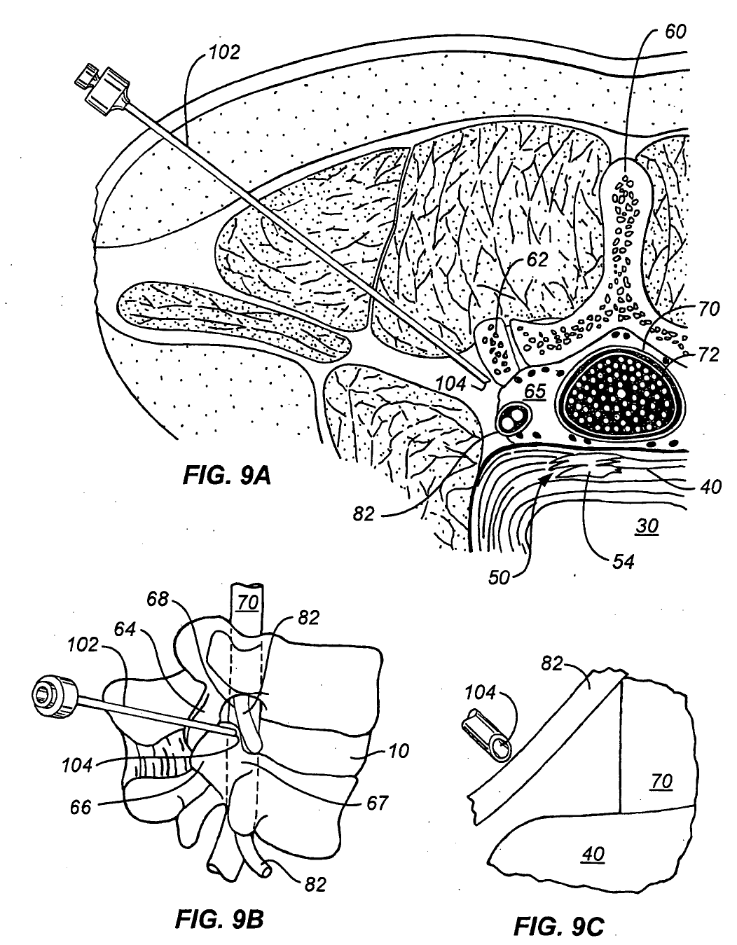

FIGS. 9A to 14C illustrate a first exemplary torn annulus therapy using an embodiment of a spinal access and therapy device of the present invention. As discussed above with regard to treatment of a ruptured annulus, the first step is the approach with the trocar 102 to a position near the injury site (FIGS. 9A, 9B and 9C). In this illustrative embodiment, the trocar 102 is advanced towards the injury site 50 (i.e., torn annulus 54) using a posterolateral approach directed towards the extra foramenal access to the interspinal epidural space (best seen in FIG. 9B).

Next, the spinal access tool 110 is advanced through the trocar lumen 108, into the epidural space 65 (FIGS. 10A, B and C). As described above, the advantageous design of the spinal access device allows the surgeon tactile, direct visual reference to guide to approach the injury site and position the device 110 to initiate therapy. The atraumatic tip 118 is used to deflect a spinal nerve root 82 without injury as the spinal access tool distal end 112 is advanced towards the annulus 40. While this example illustrates the atraumatic deflection of the nerve root 82, it is to be appreciated that a surgeon may utilize the direct visualization capabilities of the device 110 to completely avoid or minimize contact with the nerve root 82. Moreover, the direct visualization capabilities of the spinal access device allow a surgeon to steer between the nerve root 82 and the dura 72 without contacting or disrupting either.

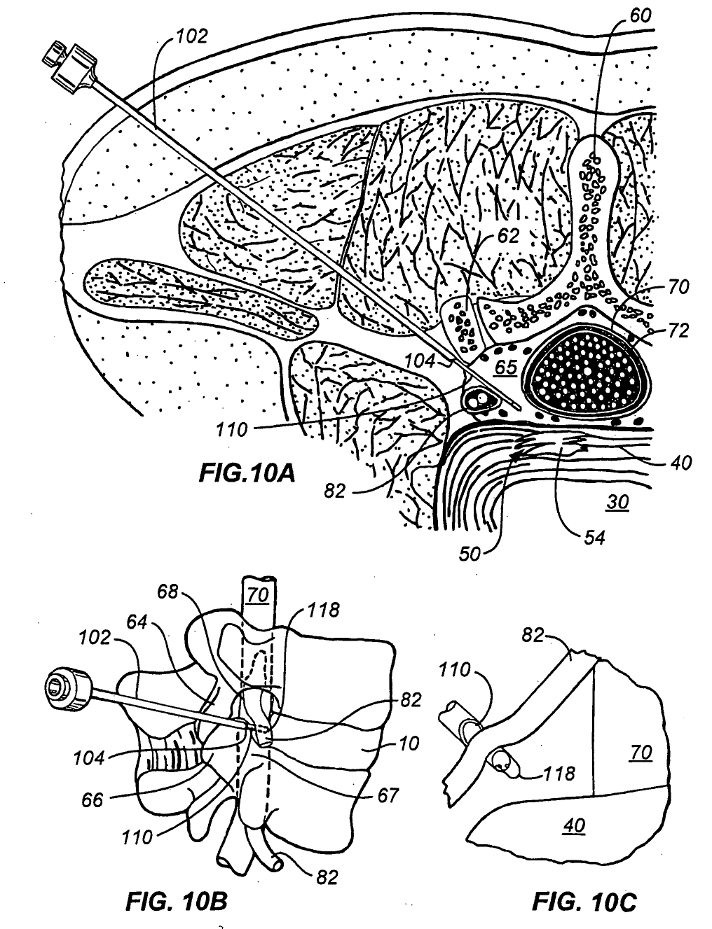

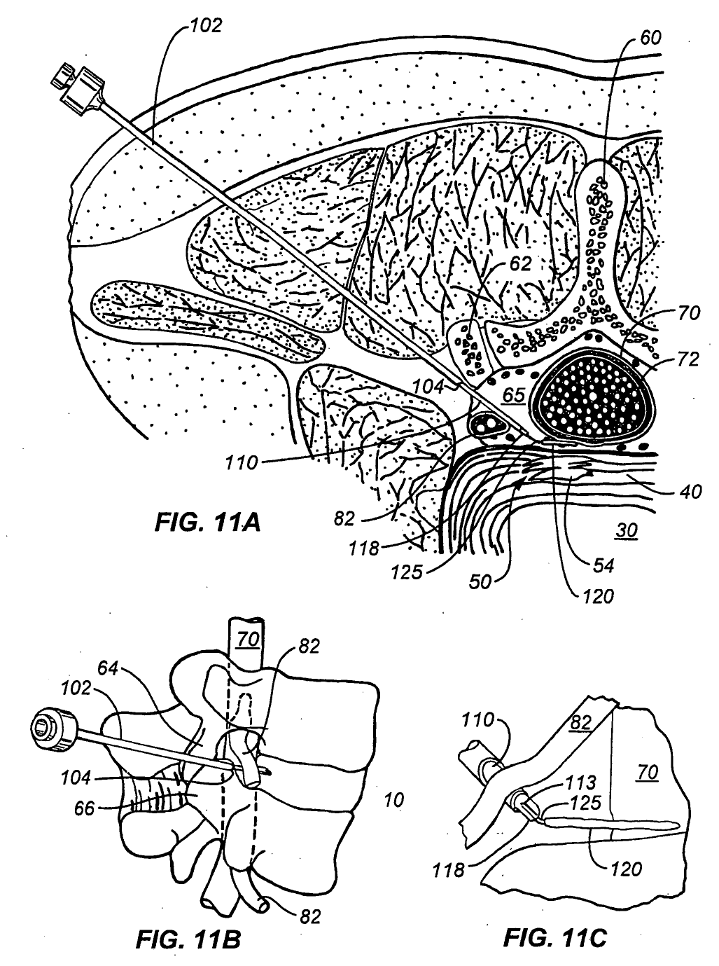

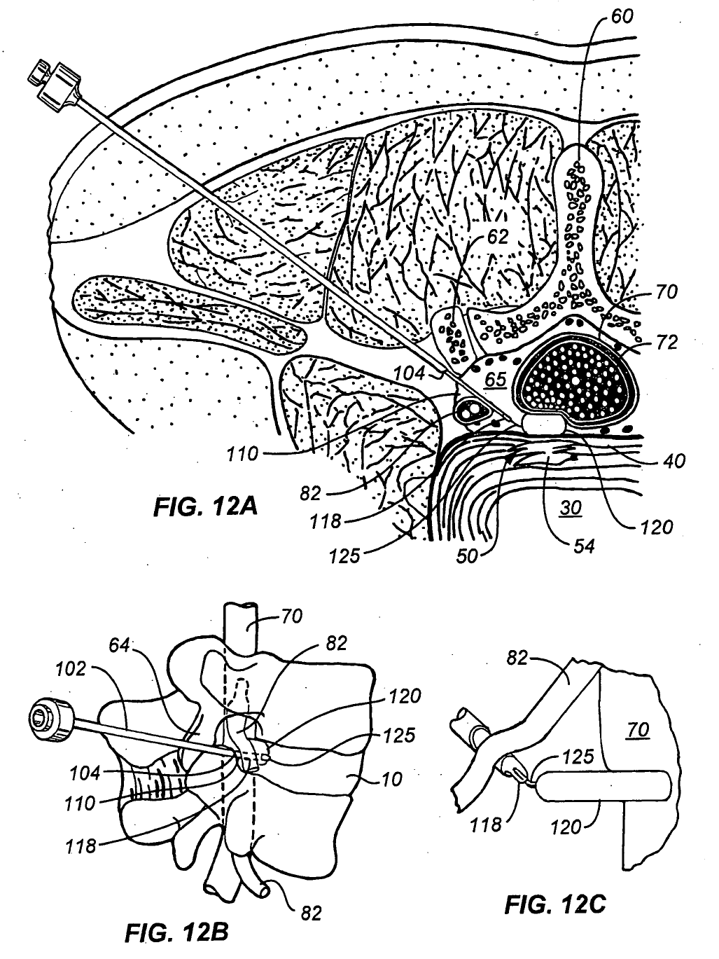

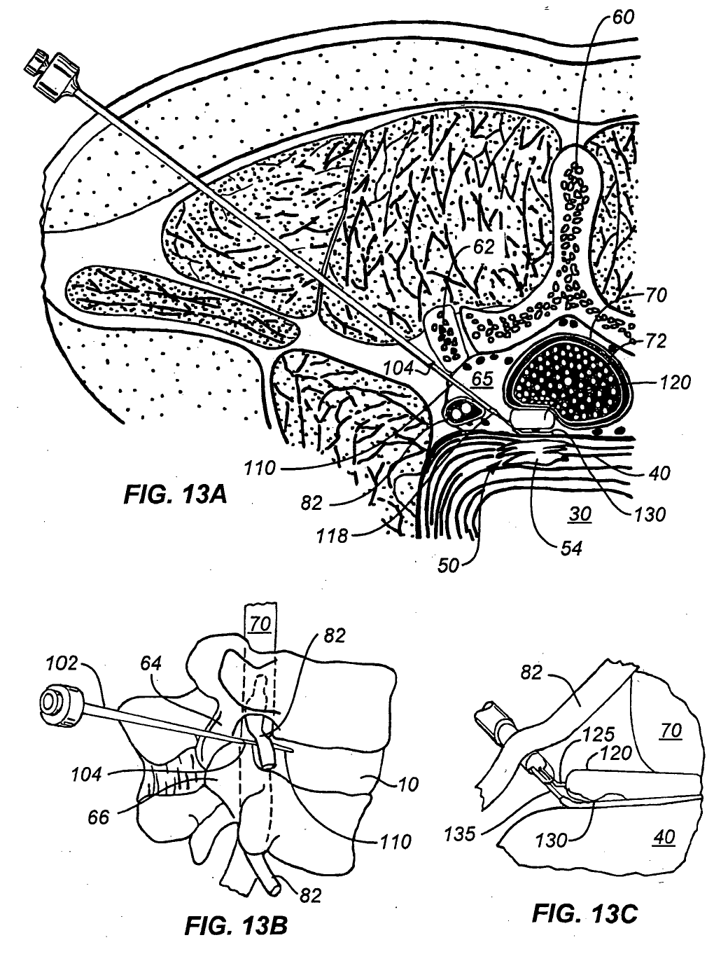

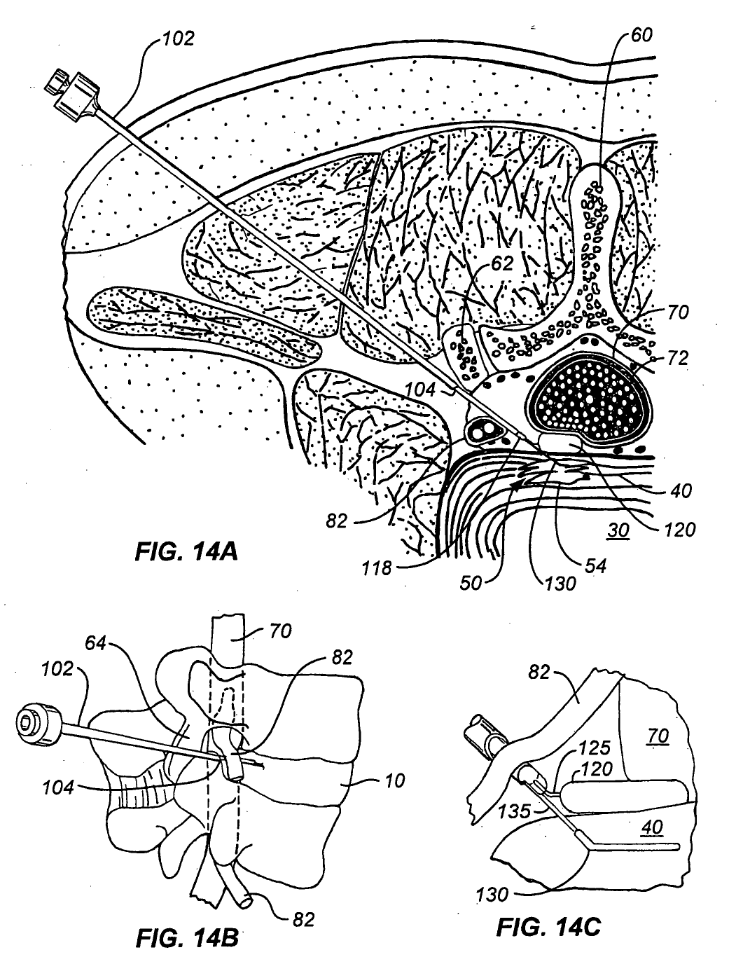

Next, as described above, the atraumatic manipulation device 120 is positioned in a stowed configuration (FIGS. 11A, 11B and 11C) and then deployed to create a therapy site or work site from which to delivery therapy or treatment (FIGS. 12A, 12B, and 12C). Next, a therapy device 130 is delivered to the therapy site to provide therapy along the posterior annulus 43 (FIGS. 13A, 13B and 13C) or within the annulus or nucleus (FIGS. 14A, 14B, and 14C).

Second Exemplary Torn Annulus Treatment

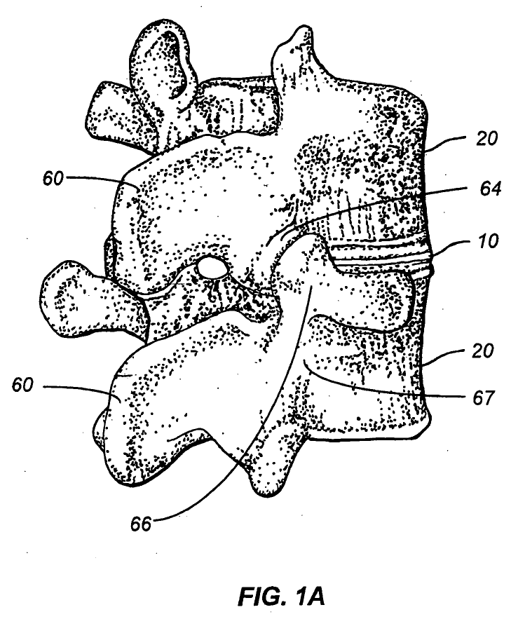

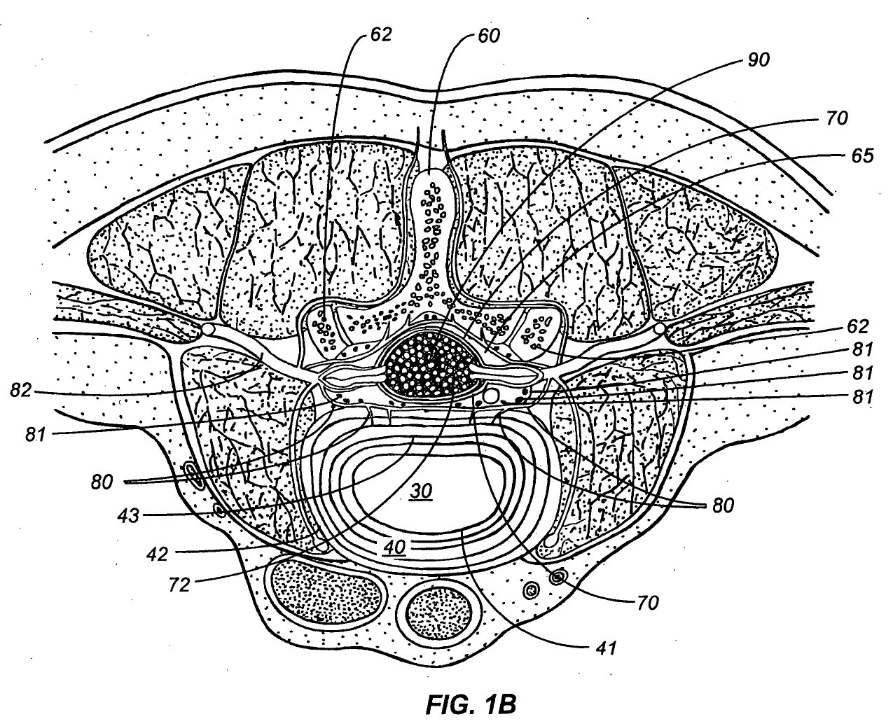

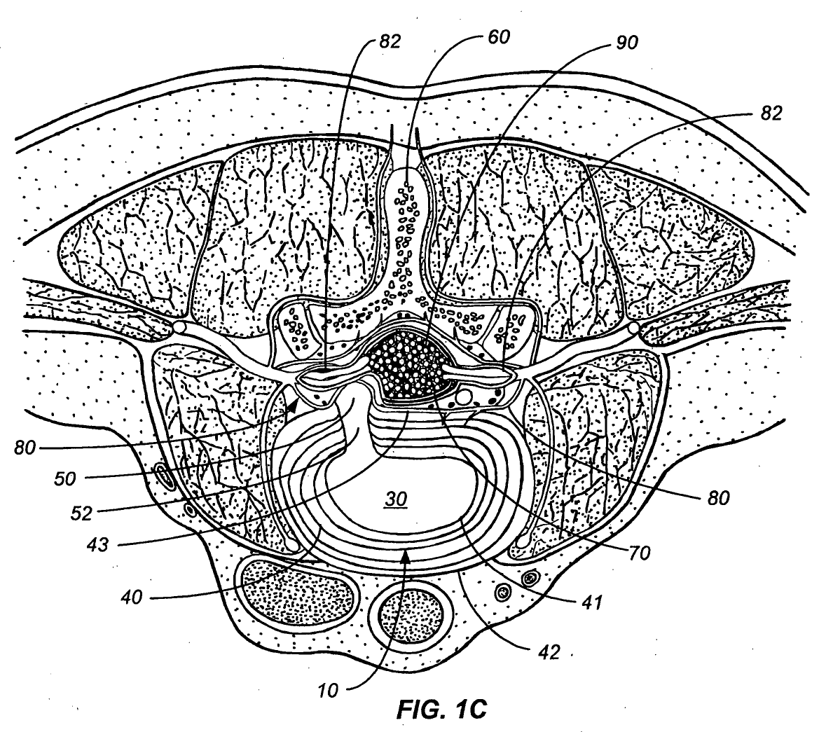

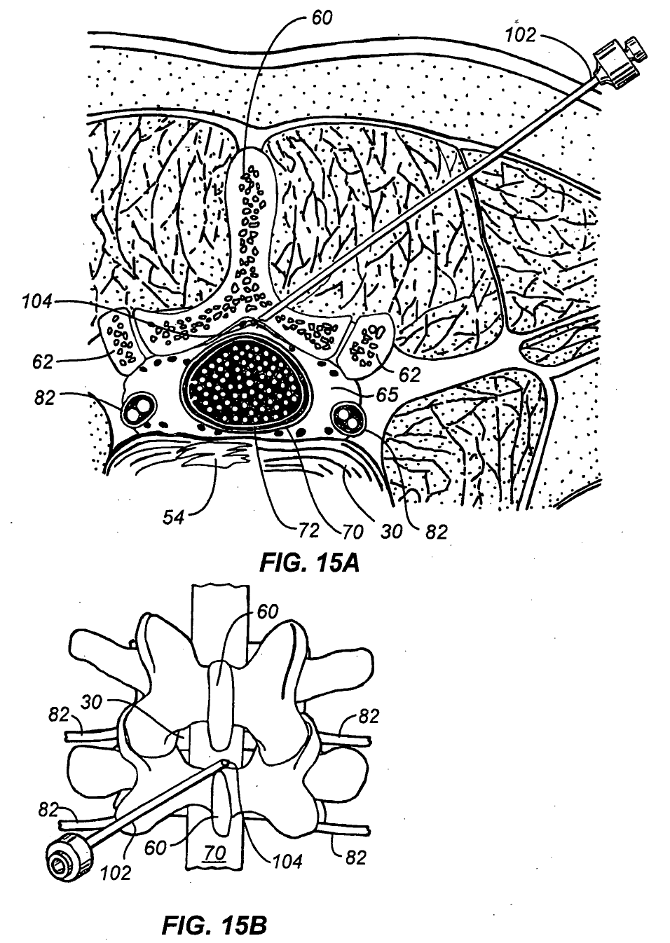

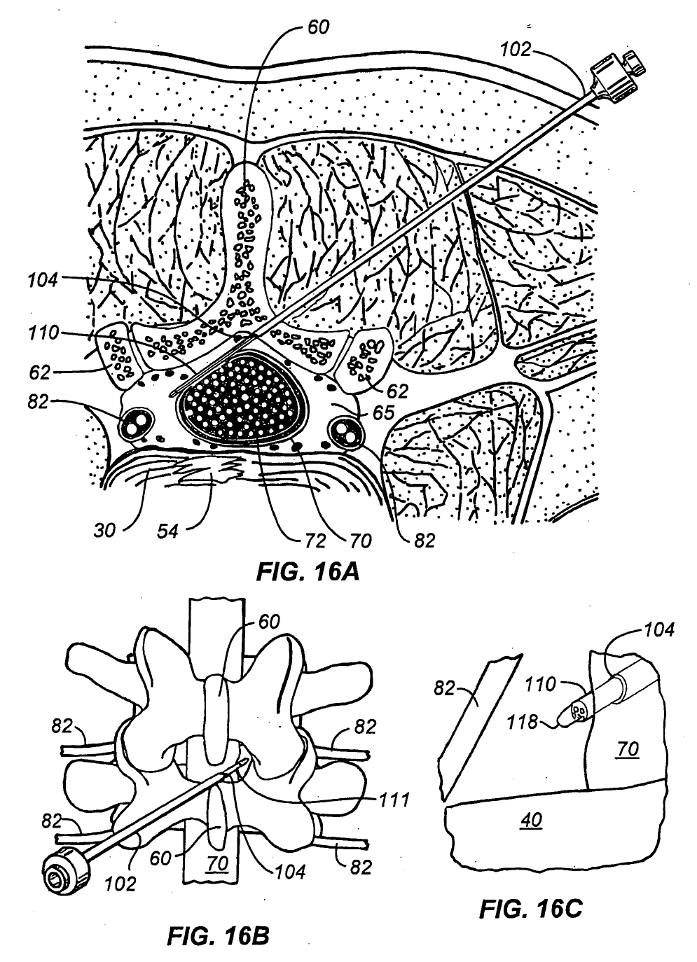

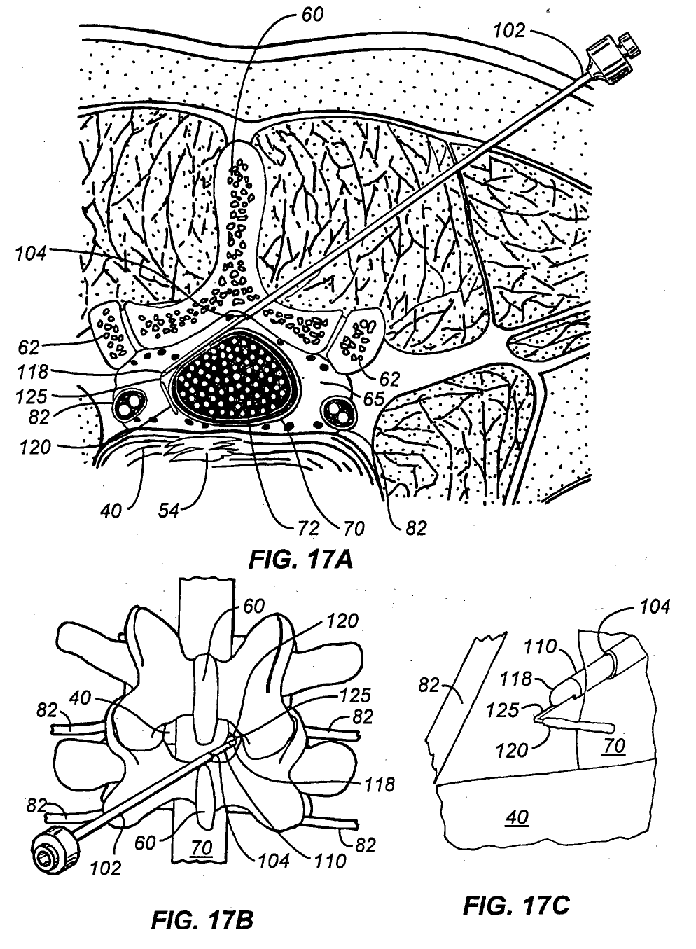

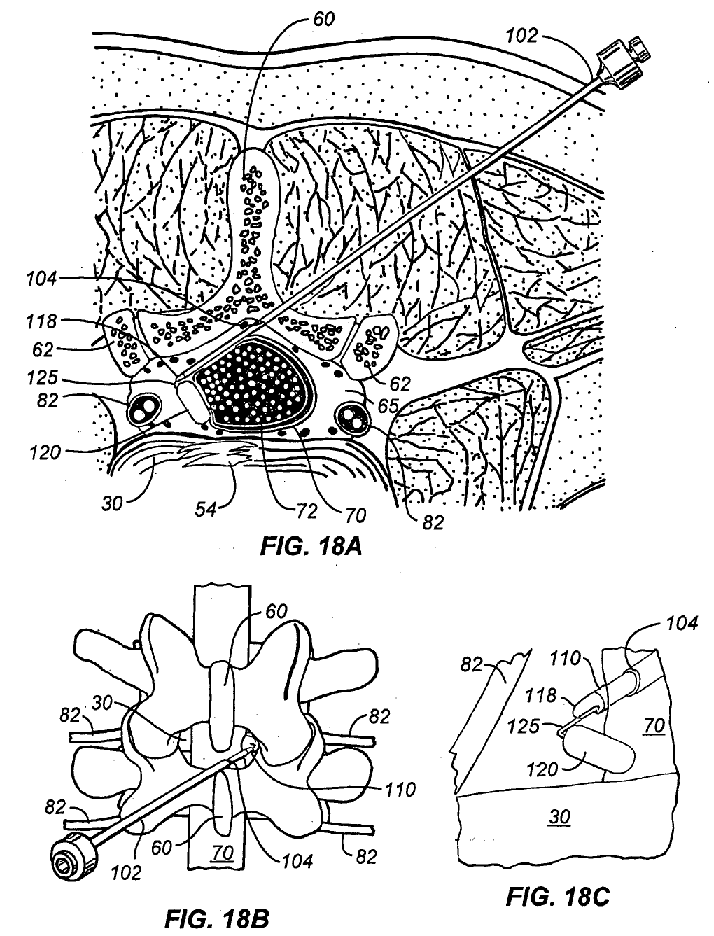

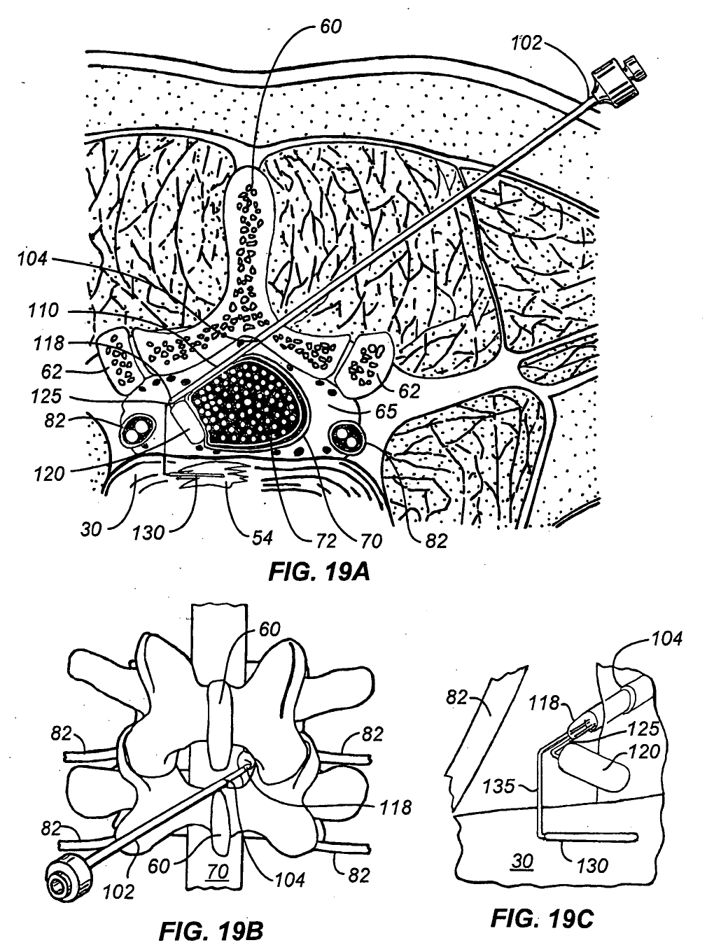

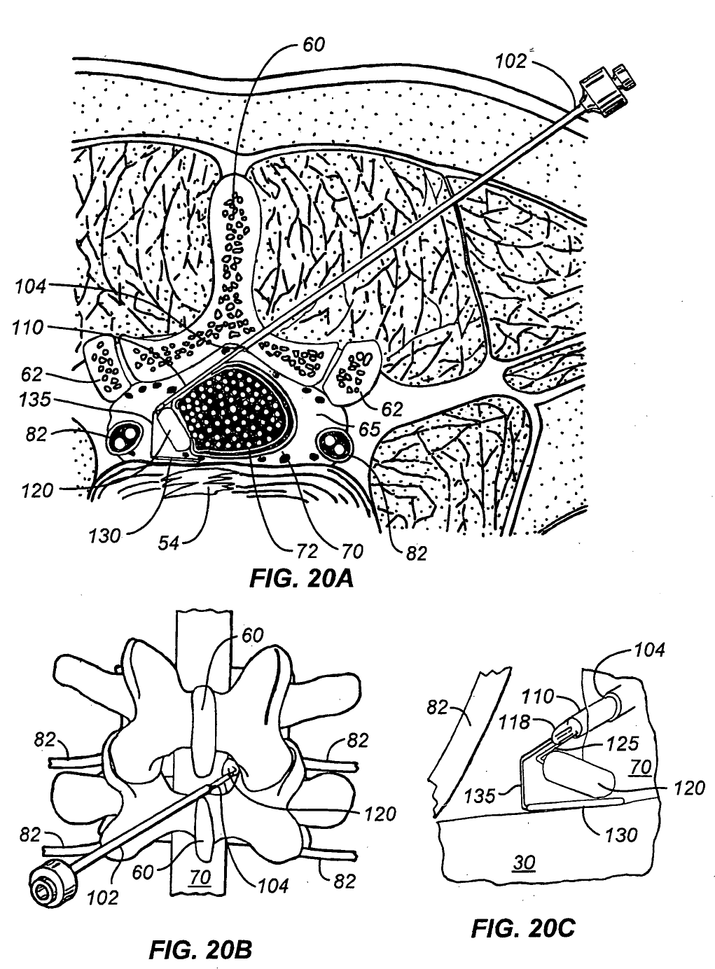

FIGS. 15A-20C illustrate a second exemplary torn annulus therapy procedure. Similar to the approaches described above with regard to FIGS. 3A, 3B and 3C, the trocar 102 is introduced in a posterolateral approach. In this illustrative embodiment, the trocar distal end 104 remains outside of the epidural space 65. Next, the spinal access tool 110 is used to advance through the epidural space 65, deform the dura 70 and position the access tool distal end 112 into position for placement of the atraumatic manipulation device 120 (FIGS. 16A, 16B and 16C). Similar to the above described procedures, the atraumatic manipulation device 120 is introduced in a stowed configuration (FIGS. 17A, 17B and 17C) and then deployed (FIGS. 18A, 18B and 18C). Thereafter, a therapy device 130 is provided below the annulus posterior surface including the nucleus (FIGS. 19A, 19B and 19C) or on the posterior annulus surface (FIGS. 20A, 20B and 20C). When the therapy probe 130 is positioned along the surface or within the first few layers of the annulus (e.g., FIGS. 20A-20C and 13A-13C), energy from the probe 130 may be used to denervate the annulus (i.e., destroy the annular nerve fibers 80 in FIG. 1B). Although illustrated and described second after placement of the probe 130 within the annulus, it is to be appreciated that this step may be performed first or that either step may be performed alone.

Alternative Spinal Access Device Embodiment

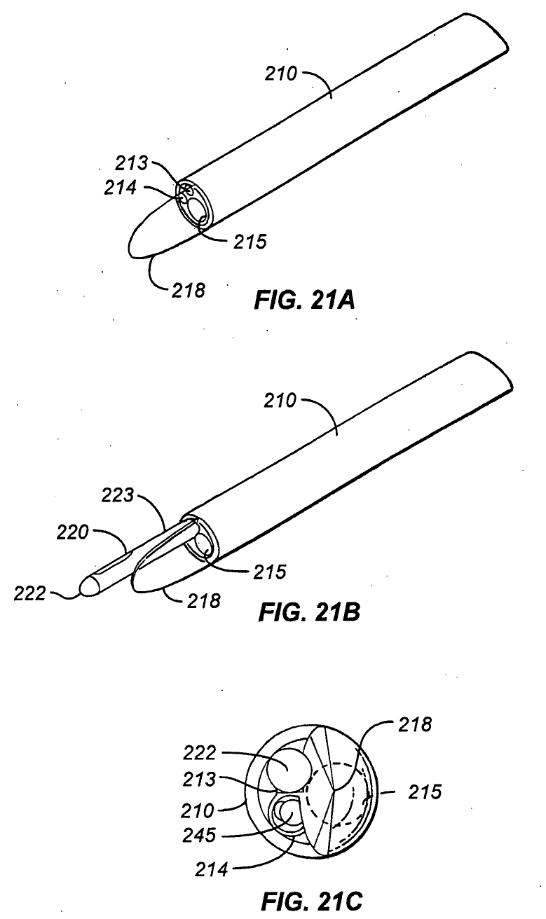

FIGS. 21A-21C illustrate an alternative spinal access device embodiment 210. The spinal access device 210 is similar in operation and appearance to spinal access device 110. As seen best in FIG. 21A, the spinal access device 210 has a visualization port 215 and atraumatic tip 218 similar to visualization port 115 and atraumatic tip 118. Also similar to device 110, the device 218 has two working channels 213 and 214. Similar to atraumatic tip 118, atraumatic tip 218 is transparent to the visualization and imaging means used in visualization port 215 and may also be rigid, inflatable or adapted for controlled deflection (i.e., to nudge adjacent tissue). As best seen in FIG. 21B, the atraumatic manipulation device 220 is stowed in a catheter 223 positioned in the upper working channel 213. The atraumatic manipulation device 220 has a rounded distal end 222. The atraumatic manipulation device 220 may also act as a shield when a therapy device 245 is provided via working channel 214. Advantageously, a therapy device 245 introduced via working channel 214 moves independent of the shield/manipulation device 220 provided via the working channel 213, and vice versa. The same advantage is also available for embodiments of spinal access device 110.

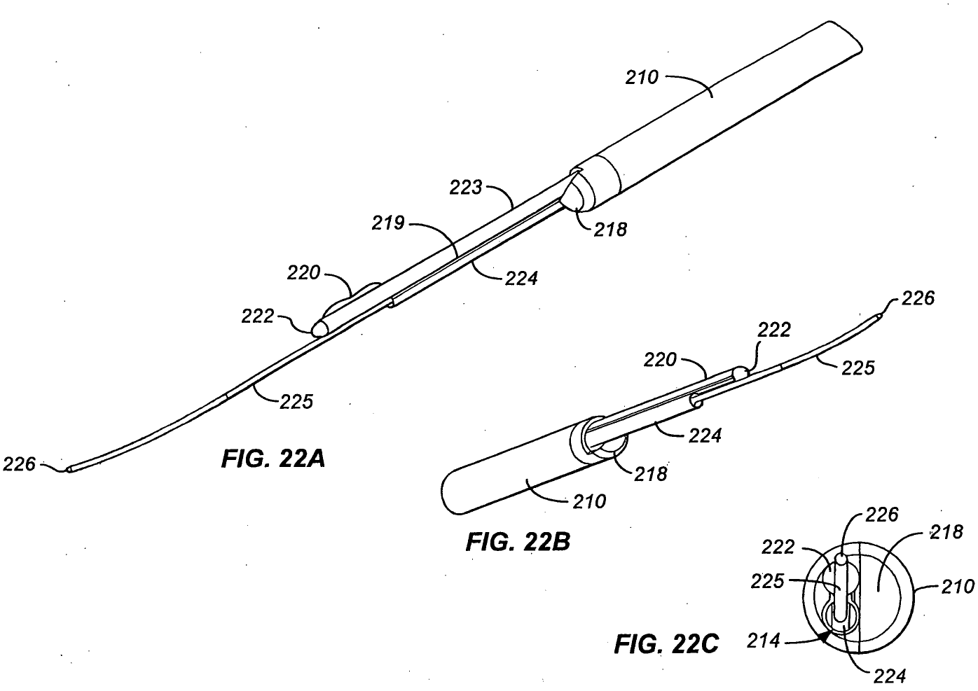

FIGS. 22A-22C illustrate yet another advantage of embodiments of the present invention—the use of a guide wire 225 for spinal guidance. In one illustrative embodiment, an integrated, flexible spinal access device 219 is used in conjunction with a guide wire 225. The integrated, flexible spinal access device 219 includes a lower working lumen 224 joined to an upper working lumen 223. In this embodiment, the distal end of the lower lumen 224 is proximal to the distal end of the lumen 223. Also illustrated in this embodiment, the upper lumen 223 has a distal end 222 and carries an embodiment of a protection device 220. The protection device 220 is illustrated in a stowed configuration that protrudes above the surface of the lumen 223. The distal end of the lumen 224 is positioned proximal to the distal end of the lumen 223 and distal to the proximal end of shield 220. In this manner, a therapy device from lumen 224 may be readily positioned adjacent to but independent of the shield device 220. A guide wire 225 dimensioned to fit within the lower channel 224 is advanced along the lower channel 224 into the spinal space until the guide wire distal end 226 is maneuvered into the desired position. Thereafter, an embodiment of the integrated, flexible spinal access device 219 is advanced along the guide wire 225 and into position. In one embodiment, the integrated, flexible spinal access device 219 is formed from materials that are more flexible than the guide wire 225 such that the advancing integrated, flexible spinal access device 219 takes the shape or adopts the curve or position of the guide wire 225. Moreover, the integrated, flexible spinal access device 219 is flexible enough so as not to disturb the placement of the guide wire 225 within the spinal space.

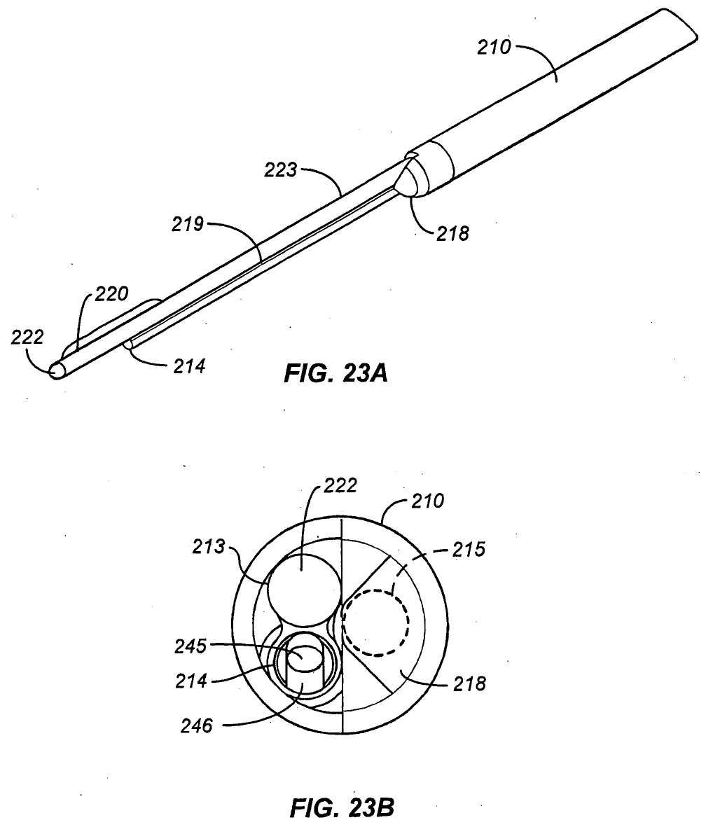

It is to be appreciated that the guide wire 225 and techniques for guide wire placement are similar to those used in other surgical disciplines. As such, the guide wire 225 may also be a steerable guide wire in some embodiments. Similar to other guide wire procedures using over the wire exchange, integrated, flexible spinal access device 219 is advanced into position by passing over the guide wire (FIG. 23A) within the spinal space. Next, the lower channel 224 used to guide in the therapy device 245 on a delivery catheter 246 (FIG. 23B). As such, the guide wire is positioned adjacent spinal structures or anatomy to provide a pathway or pathways for spinal access device embodiments of the present invention.

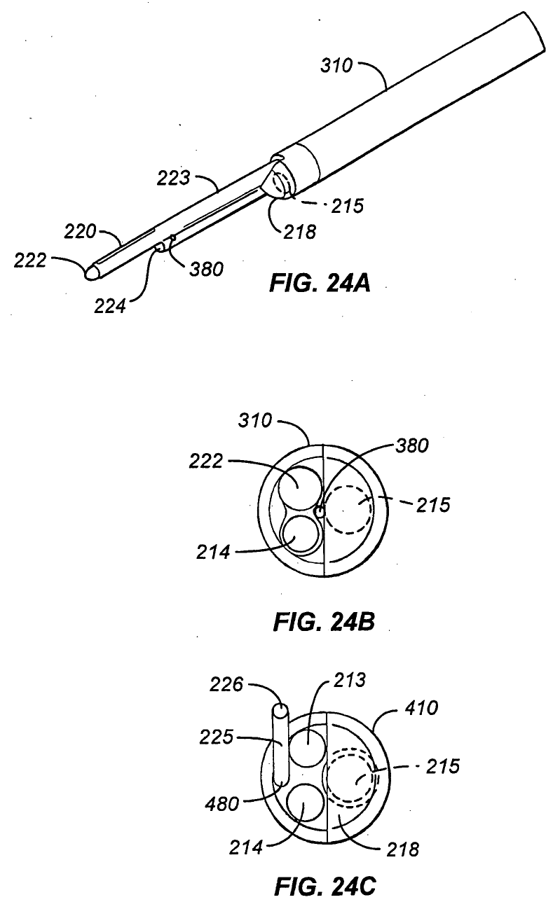

In contrast to FIGS. 22A-22C where a guide wire is used in a working channel, the next spinal access device embodiments provide a dedicated guide wire lumen. In an alternative embodiment, a spinal device 310 provides a dedicated guide wire lumen 380 (FIG. 24A). The spinal device 310 is similar the spinal device 210 with the addition of the lumen 380 on the same side of the device as the atraumatic tip 218 as best seen in FIG. 24B. Additionally, and in contrast to FIG. 22A, the stowed shield 220 illustrated in the embodiment of FIG. 24A is recessed below the surface of the lumen 223. In another alternative embodiment, a spinal device 410 provides a dedicated guide wire lumen 480 on the side of the device opposite the atraumatic tip 218 (FIG. 24C). Additionally, spinal access device 410 illustrates an embodiment where completely separate lumens 213, 214 are provided. However, partially or completely joined lumen designs are possible such as those illustrated in FIG. 24B or FIG. 22C, for example.

Other guide wire lumen, working channel, visualization port and atraumatic tip configurations are possible. In one embodiment, the embodiment of the spinal device is more flexible than the guide wire or, alternatively, the guide wire is more rigid than the embodiment of the spinal device so that the guide wire will remain in or near the desired position as the spinal device embodiment is advanced. In one aspect, the position of the guide wire within the spinal space is observed during spinal access tool advancement to confirm that the guide wire position remains in a desired position. In another aspect, guide wire positioning observations may be performed using a conventional external imaging modality such as, for example, fluoroscopy or MRI.

Third Exemplary Torn Annulus Treatment

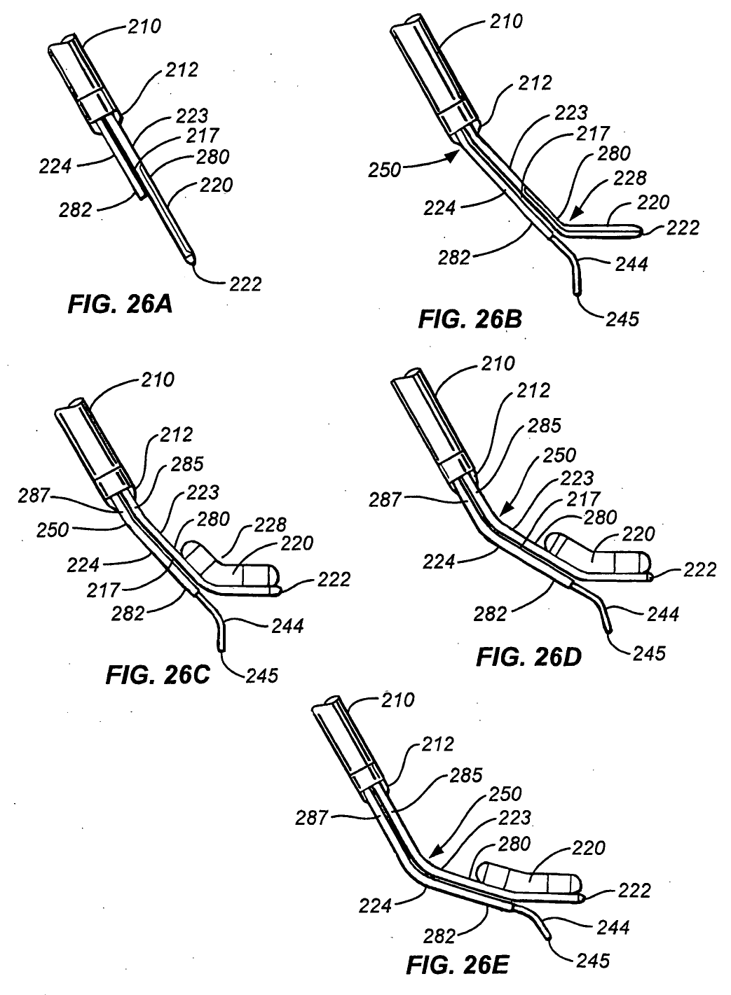

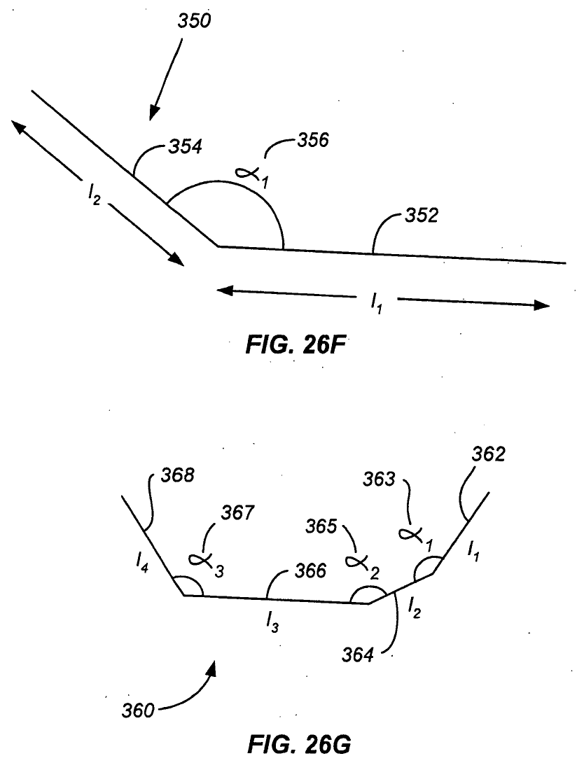

A third exemplary torn annulus treatment will now be described with reference to FIGS. 25A-26E. As described above, the steerable spinal access device 210 is introduced or otherwise positioned adjacent the spinal injury. In this illustrative example, a posterolateral approach similar to that described above is used (i.e., see FIGS. 9A-14C). FIGS. 25A, 25B and 25C illustrate the lateral advancement of the spinal therapy device and shield to three therapy positions, a first therapy position 92 (FIG. 25A), a second therapy position 94 (FIG. 25B) and a third therapy position 96 (FIG. 25B). FIGS. 26A-26E illustrate the relative positions of the spinal access device components as the therapy progresses from therapy position 92 (FIG. 25A/FIG. 26C), therapy position 94 (FIG. 25B/FIG. 26D) and therapy position 96 (FIG. 25C/FIG. 26E). The number and placement of therapy positions will depend upon the pathology being treated, the type of therapy used, and the specific anatomical make-up of the patient among other things.

As illustrated in FIGS. 26A-26E, the shield delivery catheter 223 and the device delivery catheter 224 may be separate structures that move separately or together in the spinal region. However, other delivery catheter configurations are possible. For example, the catheters may be separate but moved together as in the case where the catheters are joined outside the spinal access device and/or advanced simultaneously through the working channels. In another aspect, the catheters may be a joined together or only partially moveable relative to one another (see, e.g., FIGS. 25G-L).

Returning to the illustrative embodiments in FIGS. 25A, 25B and 25C, there is an annular tear 54 that will be treated by penetrating an energy probe 245 into the annulus 40 in three therapy positions. It is to be appreciated that prior to performing the therapies illustrated in FIGS. 25A, 25B and 25C, the probe 245 may surface treat or penetrate the annulus 40 to denervate the annulus 40 (i.e., destroy the annulus nerve roots 80 in FIG. 1B). Moreover, successful denervation may advantageously be accomplished using a set of therapy positions. Progression across the posterior annulus region may be assisted through the use of the shield/manipulation member 220. For example, from the first therapy position 92 to the second therapy position 94 the shield 220 could be transitioned back to a stowed condition and advanced towards the second therapy position using the distal tip 222 to manipulate tissue or partially deploy to move tissue. Once in the desired position adjacent the second therapy position 94, the second catheter 224 with therapy probe or instrument 245 is advanced to the second therapy position 94. Before or during the second catheter 224 advancement or prior to initiating therapy in the second position 94, the shield/manipulation device 220 transitions to the deployed configuration (FIG. 25B). Alternatively, the shield/manipulation device 220 could also remain in the deployed configuration or partially deployed configuration as it advances from one therapy position to another.

FIGS. 25D-25I illustrate that while the shield 220 is positioned in one of several spinal therapy positions, such as positions 92, 94, a therapy device may be positioned in one or more of a plurality of application positions adjacent to or within a spinal area shielded by the shield 220. In FIGS. 25D, 25E and 25F the shield 220 remains in a constant therapy position within the spinal space. The therapy device 245 however is introduced into or provides therapy to the annulus 40 adjacent the annular tear 54 in the application positions 2502, 2504 and 2506. Note that application positions proceed from a proximal to distal position relative to shield 220. The therapy probe 245 may be provided into more or different application positions than those illustrated or in any order.

FIGS. 25G, H and I illustrate the combination of multiple different therapy positions (i.e., where is the shield positioned in the spinal space) and multiple different application positions (i.e., where is the therapy probe 245 position relative to the shield 220 or shielded spinal portion). In this embodiment, the shield 220 is attached to the shield delivery catheter 2523 that is delivered via a working channel in device 2587. Device 2587 is a simplified view of an embodiment of a spinal access device 210 for purposes of discussing these aspects of the invention. The device delivery catheter 2524 moves independent of the shield 220 and the shield delivery catheter 2523. As indicated in phantom and solid representations of the distal end of the device delivery catheter 2524, this movement is used to position the pre-formed therapy device 245 into application positions 2502, 2504 and 2506. In this illustrative embodiment, the application positions 2502, 2504, and 2506 are in a constant relationship to the shield 220. This need not be the case and other application positions are possible.

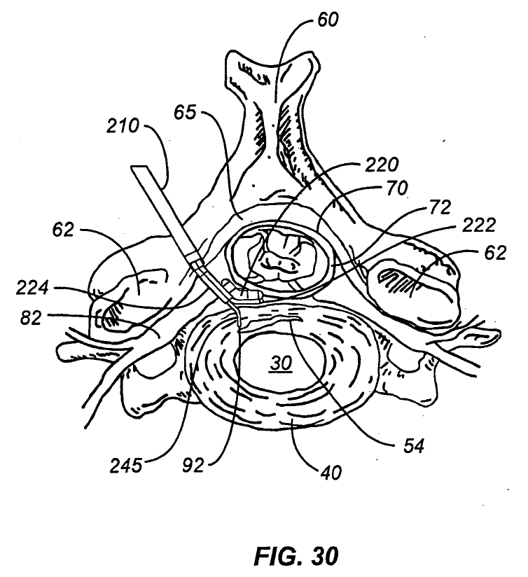

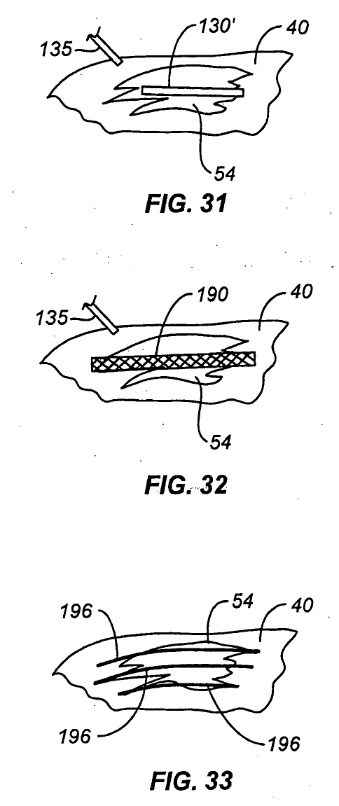

Distal movement of the catheters 2523, 2524 moves the shield 220 between different therapy positions (FIGS. 25G, 25H, and 25I). In each therapy position, the device delivery catheter distal end moves to provide placement of the therapy device 245 into the illustrated therapy positions 2502, 2504, and 2506. For example, the shield 220 is advanced (either in a stowed, deployed or partially deployed configuration) into a first therapy position in the spinal space (FIG. 25G). Next, with the shield 220 providing shielding to adjacent structures (as illustrated by shield 220 placement in FIG. 30), an embodiment of the therapy device 245 is provided via the device delivery catheter 2524 into the shielded space provided by shield 220 into one or more application positions. In these illustrative embodiments, the distal end of the device delivery catheter 2524 slides along or, alternatively, is moved independent of the shield delivery catheter 2523 into three application positions 2502, 2504 and 2506. While three therapy/application positions are illustrated by FIGS. 25G, 25H and 25I, more or fewer therapy/application positions may be used. The application positions may be in any number of different orientations relative to the shield 220 based, in some embodiments, on the pre-formed shape of the therapy device 245. As such, it is to be appreciated that the therapy device 245 may be provided in orientations other than the generally orthogonal relationship illustrated in these embodiments. The process above repeats as the shield 220 is advanced into second and third therapy positions as illustrated, respectively, in FIGS. 25H and 25I. It is to be appreciated that the illustrative embodiments of FIGS. 25E-25I may also include aspects of a pre-shaped, integrated, flexible spinal access tool (i.e., the channels 223/224 move together) or a pre-shaped, independent, flexible spinal access tool (i.e., the channels 223/224 may move independent of one another) embodiments of the spinal access tool of the present invention.

In an alternative embodiment, multiple application positions are provided using an extendable or telescopic member 2588 (FIGS. 25J, 25K and 25L). In this embodiment, the distal end of the device delivery catheter 2524′ is fixed relative to the shield delivery catheter 2523. The extendable member 2588 is housed in and moves relative to the device delivery catheter 2524′. Similar to FIGS. 25G, 25H and 25I, the shield 220 is positioned in first, second, and third therapy positions in FIGS. 25J, 25K and 25L, respectively. FIG. 25J illustrates a therapy device 245 in a first application position 2502 relative to the shield 220. In the first application position 2502, the extendable member 2588 is not used to position the therapy device 245. In other words, the extendable member 2588 is not extended beyond the distal end of the device delivery catheter 2524′. The extendable member 2588 is extended distally from the device delivery catheter 2524′ to reach a second application position 2504 (FIG. 25K). The extendable member 2588 is extended still further distally to reach a third application position 2506 (FIG. 25L). It is to be appreciated that more or fewer application positions are possible and that the extendable member 2588 may be positioned in any number of positions adjacent to shield 220 and/or along the shield delivery catheter 2523. As described above, the shield/balloon 220 is moved into a desired therapy position within the spinal region and the therapy probe 245 is moved into one or more application positions to provide therapy to the spinal region. In these embodiments, the location of the application position is determined by the position of the extendable member 2588 relative to the device delivery catheter 2524′.

Alternatively, embodiments of the spinal access device of the present invention may also enable denervation procedures to be performed as a separate procedure using direct visualization from the spinal access device. The approaches used for denervation may be similar those described herein to access the posterior annulus. It is to be appreciated that the denervation procedures may be performed to relieve discogenic pain and/or before the disc damage has progressed to a herniated disc or torn annulus.

FIGS. 26A-26E illustrate an embodiment of a pre-shaped spinal access device 217. In this embodiment, the shield channel 223 and the therapy probe channel 224 are integrally formed into a single device 217. Unlike alternative embodiments where relative movement between the channels 223, 224, probes and shields, the channels 223, 224 in this embodiment move as a unitary body. The shield channel has a distal tip 222 and a stowed shield 220. In this embodiment, the shield 220 is at or below the outer surface of the shield channel 223 when in a stowed configuration. In other embodiments, the stowed shield 220 may extend above the surface from a recessed position in the shield channel 223 (e.g., FIG. 23A) or be mounted on the surface of shield channel 223.

FIGS. 26A-26E illustrate further distal advancement of the pre-shaped spinal access device 217 from the device 210. The distal end of the shield channel 280 and the distal end of the therapy probe channel 282 have exited the treatment apparatus distal end 212 (FIG. 26A). The preformed angle 250 is exiting the distal end 212 and the full length of the first sections 280/282 are visible (FIG. 26B). Next, the preformed angle 250 is clear of the distal end 212 and the distal end of the next sections 285, 287 are visible (FIG. 26C), partially exited (FIG. 26D) and fully exited (FIG. 26E). FIGS. 26C, 26D and 26E also illustrate an embodiment of the shield 220 in a deployed configuration with a therapy probe 245 within the therapy probe channel 224. This embodiment of the therapy probe 245 also illustrates a therapy probe pre-formed portion 244. As illustrated in FIGS. 26C-26E, the pre-formed portion 244 may place the probe distal end in a different position depending upon a number of factors such as the pre-formed shape, the length of the probe 245 advanced beyond the probe channel 224, and the position of the pre-shaped device 217.

Fourth Exemplary Torn Annulus Treatment

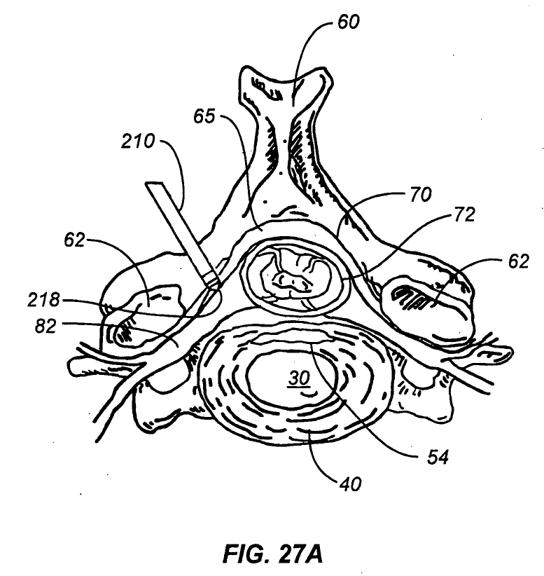

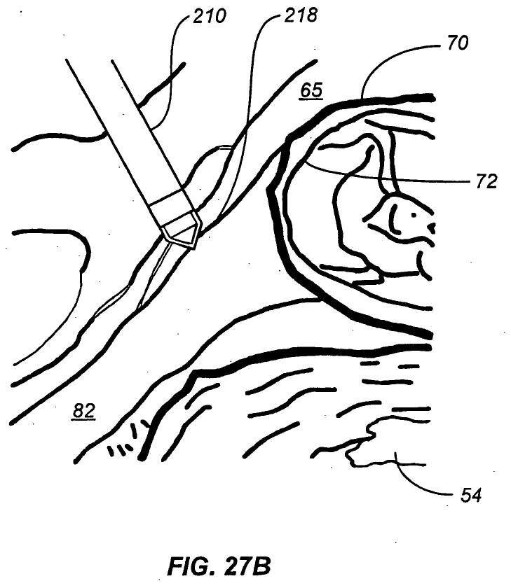

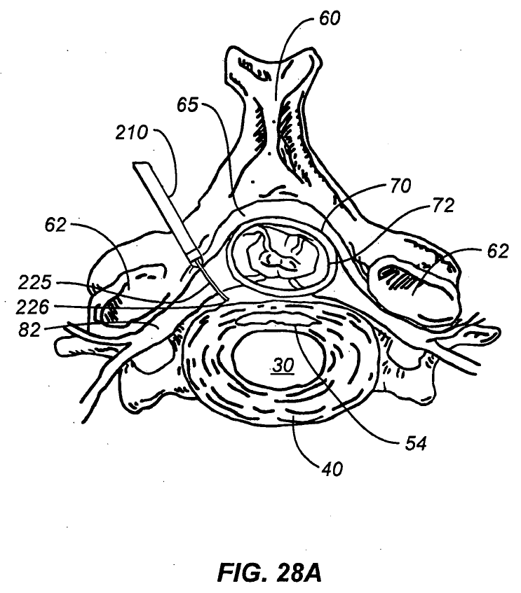

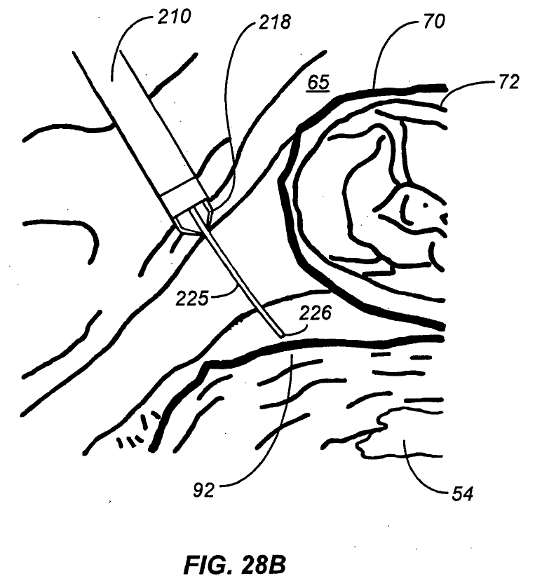

A fourth exemplary procedure will now be described with reference to FIGS. 27A through 30. This procedure is similar to the procedure described above with regard to FIGS. 25A-26E except that this illustrative procedure uses a guide wire to aid in positioning the spinal access device or components in the spinal space. Similar to other procedures, the spinal access device 210 is advanced towards the treatment area (here, a tear 54) alone, through the use of a trocar or introducer or through the use of other endoscopic techniques (FIGS. 27A and 27B). Next, a guide wire 225 is advanced along the spinal device 220 into the spinal space. The guide wire distal tip 226 is positioned in the spinal space in position for therapy or in a position to advance the device 220 or portion thereof (FIGS. 28A and 28B). While illustrated as being introduced through a spinal device 220 (i.e., where guide wire 225 is introduced using a working channel), it is to be appreciated that spinal access devices having a dedicated guide wire lumen may also be used (e.g., spinal access device 310 in FIG. 24A or spinal access device 410 in FIG. 24C).

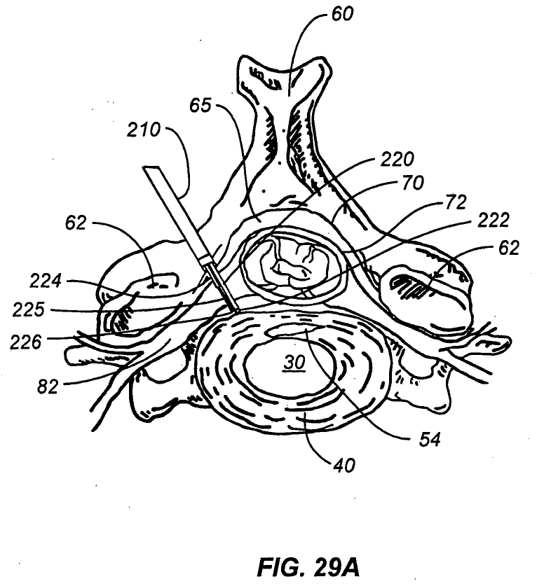

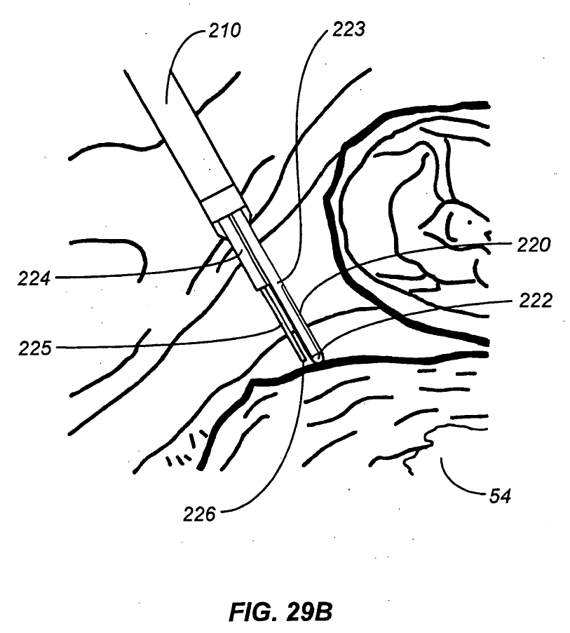

Next, the upper and lower working channels 220, 224 are advanced along the guide wire 225. As best seen in FIG. 29B, the guide wire 225 was positioned in the lower channel 224 in this illustrative embodiment. At this point, the guide wire 225 could be further advanced along the annulus to another position and then the device would advance again. The process of alternately advancing the guide wire 225 and then advancing the working channels 220, 224 repeats until the desired therapy position is reached. Once in the desired therapy position, the guide wire 225 is with drawn and the therapy device is introduced, the balloon/shield 220 deployed and the therapy performed (FIG. 30). Thereafter, the therapy device is withdrawn, the guide wire 225 re-introduced into the working channel and then advanced to the next desired position. Then using the guide wire as described above, the working channels are advanced using the guide wire 225. This process repeats until the next therapy position is reached when another guide wire/therapy device exchange is performed. Any number of positions and types of therapy may be performed as discussed above with regard to FIGS. 25A-26E. Additionally, the above description may be modified to include the use of multiple application positions and other configurations as discussed above with reference to FIGS. 25A to 25I.

The pre-shaped spinal access device 217 may have any of a number of different configurations depending upon the portion of the spinal space being accessed for therapy. Two alternative pre-shaped spinal device embodiments are illustrated in FIGS. 26F and 26G. Pre-shaped access device 350 illustrates one embodiment (FIG. 26F). Pre-shaped device 350 has a distal section 352 of length l₁ and a proximal section 354 having a length l₂. The distal section 352 and the proximal section 354 define an included angle 356 (α₁). The lengths l₁ and l₂ and the angle (α₁) may be selected depending upon the specific portion of the spinal region being accessed and the approach method employed. Pre-shaped device 360 illustrates an embodiment having four sections 362, 364, 366 and 368 having lengths l₁, l₂, l₃, and l₄ respectively (FIG. 26G). There are three included angles: angle 363 defined between sections 362, 364; angle 365 defined between sections 364, 366; and angle 367 defined between sections 366, 368. It is to be appreciated that other pre-shaped spinal access device configurations are possible. For example, the sections 352, 354, 362, 364, 366 and 368 need not be straight but may be curved or formed into other patterns. Similarly, the included angles between two sections may be angled less than 180 degrees. As these exemplary pre-shaped spinal access device embodiments make clear the distal end of the distal section (section 352 or section 362) may be maneuvered into a variety of positions through selection of angle and section length of the proximal sections and angles. Similarly, any section portion may be manipulated using the geometry of the adjacent section or sections and/or angle or angles to provide the desired advancement and positioning characteristics of the pre-shaped spinal access device.

In another alternative embodiment, the spinal access device may be used to deliver one or more annulus reinforcement elements or may have a detachable portion that becomes an annulus reinforcement element. In one specific aspect, the therapy probe 130′ is separable from the catheter 135. After insertion into the annulus 40 or within the tear 54, the separable probe 130′ is detached and remains in the annulus 40. The separable probe design or configuration may be altered to enhance its structural characteristics such that it may be effective in both the role of applying the therapy as well as post-therapy structural support. Alternatively, a separate structural support element may be provided for structural support in the spinal access area. Structural support 190 is illustrated in position in an annular tear 54 (FIG. 32). Structural support 190 is a stent-like structure dimensioned to fit within a portion of the annulus. In yet another alternative embodiment, a plurality of small or fine structural elements 196 may be provided into a tear 54 in an annulus 40 (FIG. 33). While illustrated for a torn annulus, embodiments of the structural element may be provided elsewhere within the spinal space and the design adapted for the particular injury, therapy or anatomy encountered.

Disc Augmentation Systems

The subject devices may also be used in systems for disc augmentation. A disc augmentation device may be introduced by the above spinal access device for the prevention or treatment of disc degeneration. Augmentation refers to both (1) annulus augmentation which includes repair of a herniated disc, support of a damaged annulus, closure of a torn annulus and (2) nucleus augmentation in which additional material is added to the nucleus.

Annulus augmentation devices are implanted in order to treat, delay or prevent disc degeneration. The implanted augmentation device provides structural support and absorbs at least part of the mechanical loads exerted onto the annulus. This annular fortification prevents or reduces rents, fissures and subsequent herniations. In addition, these devices reduce the pressure on the nerves which often leads to pain, weakness and/or numbness in the lower extremities, upper extremities, or neck region.

Various annulus augmentation devices include meshes, cages, barriers, patches, and scaffolds. The annulus augmentation devices may also be used to close a defect in the annulus. For example a flexible barrier material, such as Dacron™, may be affixed to the annulus to seal the annulus defect. The barrier is affixed by using sutures, staples, glues or other suitable fixation means well known in the art.

Nuclear augmentation devices add material to the nucleus in order to restore diminished disc height and pressure as well as rehydrate the nucleus, thereby adding to its fluidity. This material may be permanent, removable, or absorbable. In some instances, nuclear augmentation devices are able to induce the growth or formation of material within the nuclear space. Various nuclear augmentation devices include liquids, gels, solids, or gases such as hydrogels, silicones, or growth factors.

The subject devices improve delivery of an augmentation device because of the direct visualization capabilities and the creation of a working area. Furthermore, the creation of a working area clears an area for improved visibility. The subject spinal access device may also introduce saline or a cleaning solution to further improve visibility within the working area. In addition, the spinal access device's atraumatic tip provides tactile feedback to the user of the rigidity, pliability or feel of the tissue or structures in contact with the tip. For example, the ability to steer the device between the nerve root and the dura increases the precision in which an augmentation device may be implanted while minimizing any pain or damage to the nerves.

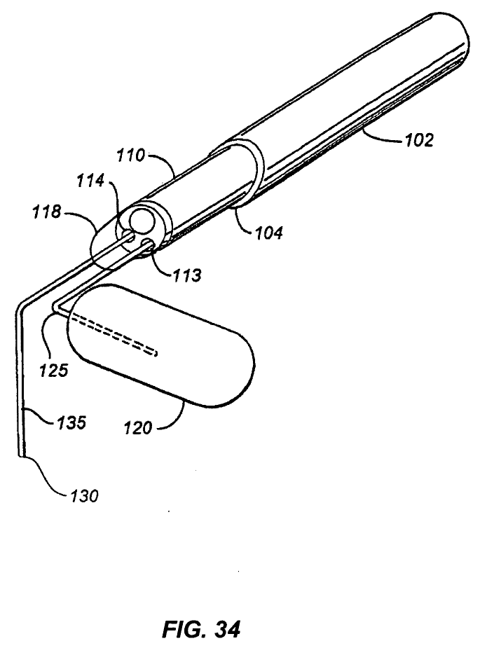

FIG. 34 illustrates an embodiment of a spinal access device 110 of the present invention used to deliver an augmentation device detachably disposed on a distal end 130 of a steerable catheter 135. The spinal access device 110 includes a pair of working channels 113 and 114 in a distal end 112. The spinal access device may further include an aspiration port or an irrigation port. Additionally, the spinal access device 110 has a visualization port covered by a shaped atraumatic tip 118. The visualization port is used by illumination, visualization and/or imaging components to provide direct visualization capabilities for implanting the disc augmentation device. The visualization port may house one or more conventional illumination, visualization, analytical and/or imaging components used to illuminate, visualize, analyze or image the surrounding anatomical environment. The spinal access device 110 is illustrated within a conventional trocar or introducer 102. The disc augmentation device would be advanced through the working channel 114 and positioned at the distal end 130 of the catheter. Using the direct visualization capabilities of the subject spinal access device, the disc augmentation device would be further advanced from the distal tip (130) of the catheter and out into the working area created by the atraumatic manipulation device 120 alone or in combination with the atraumatic tip 118.

In one aspect of the invention, the manipulation device remains in place while the augmentation device is being placed and deployed within the disc. In another aspect, the manipulation device may remain in a deployed state and detached from the catheter. In this way, the manipulation device remains in place to provide a working area. In another aspect, once the working area has been created, the manipulation device may be removed thereby allowing working channel to be used for another device, for example by providing access for a nucleus decompression device to decompress the nucleus either prior to or after re-inforcing the annulus.

In an exemplary method for introducing an augmentation device; the spinal access device is directly introduced or steered to a position adjacent the outer surface of the spinal dura matter using the visualization information provided by the instrument. Next, the spinal dura matter is displaced with the atraumatic manipulation device in order to create a working space. The atraumatic manipulation device may be partially deployed or cycled through deployed, stowed, and partially deployed positions to manipulate tissue and surrounding structures as previously described. The surgeon may also be aided in guiding the spinal device through use of direct visualization provided by the instruments in the visualization port. As such, the spinal access device is advanced using the visual or image data from the visualization port, tactile feedback from the atraumatic tip and/or external image information. Finally, the augmentation device is introduced through one of the working channels of the spinal access device. Because of the creation of a working space and the spinal access device's direct visualization capabilities; the augmentation device is then accurately positioned at the treatment site.

The augmentation device may be any device well known in the art which is suitable for repairing a herniated disc segment, supporting a weakened, torn or damaged annulus fibrosis, closing the annulus fibrosis or adding material to the nucleus pulposus, for example an ablation device. The following are a number of devices which may be used with the present invention. Each of these devices are exemplary and not to be construed as limitations.

For example, the subject device may introduce the augmentation device described in U.S. Pat. No. 6,425,919; the disclosure of which is herein incorporated by reference. The '919 device provides support for returning all or part of the herniated disc segment to a position substantially within its pre-herniated borders. The device uses an anchor as a point against which all or part of the herniated segment is tensioned so as to return the herniated segment to its pre-herniated borders, thereby relieving pressure on otherwise compressed neural tissue and structures. The elements of the '919 device is shown in FIGS. 2A and 2B of the patent in which the anchor is securely established in a location within the functional spine unit, such as the anterior annulus fibrosis shown in the figure. The device includes a support member positioned in or posterior to the herniated segment. Leading from and connected to the anchor is a connection member, which serves to connect the anchor to the support member. Tightening the connection member allows the device to transmit tensile forces along its length, which causes the herniated segment to move anteriorly, i.e., in the direction of its pre-herniated borders.

An additional exemplary device is described in U.S. Patent Application No. 2005/0070913, herein incorporated by reference. The device delivers a tissue adhesive to the inner wall of the annulus fibrosus to provide localized repair at the site of an annular fissure. A fundamental element is the injector, which applies the adhesive polymer to the treatment site. An example of the injector is depicted in FIG. 2 of the application. The device includes a handle, which holds a catheter-like compound tube which encloses an injection lumen that terminates at a distal tip. The injection device generally injects the adhesive into the interstices between the fibrous structures of the annulus, thereby becoming mechanically incorporated into the fibrous structure.

The subject spinal access devices may also be used to deliver the devices described in U.S. Patent Application No. 2005/0256582, the disclosure of which is incorporated by reference. According to the disclosure, the device includes a first end portion, a second end portion, and a bridge portion as shown in FIGS. 5A-5E of the application. The first end portion is adapted to be placed within an intervertebral body; the second end portion is adapted for placement within an adjacent intervertebral body; and the bridge portion spans a hole or defect in an annulus fibrosis.

Another exemplary device is described in U.S. Patent Application No. 2005/0240269, the disclosure of which is incorporated by reference. The augmentation device comprises a mesh frame made up of a plurality of flexible curvilinear members. One embodiment of the device is shown in FIG. 36 of the application. The device includes an enlarged central strut and a plurality of slots. The central strut can have a uniform stiffness against superior-inferior and bending. In addition, the strut can have a varying stiffness along its height to either promote or resist bending at a given location along the inner surface of the annulus.

The augmentation device in U.S. Pat. No. 6,371,990, herein incorporated by reference, is attached to the annulus fibrosis. FIG. 1B of the '990 patent illustrates how the inner wall of the annulus is reinforced through the use of a mesh stapled from within the disc space using staples. The device is made from a biocompatible fabric or mesh and is attached to the inside or outside of the annulus by stitches, staples, adhesives, or other suitable techniques. Alternatively, the fabric may be attached by screws, staples, tacks, or porous material for bone in-growth such as titanium.

Another augmentation device is described in U.S. Pat. No. 6,969,404 herein incorporated by reference. The '404 device is a collapsible bag which is inserted into the annulus fibrosis and expands to release biocompatible material such as autograft nucleus pulposus, allograft nucleus pulposus or xenograft nucleus pulposus. FIG. 1 of the '404 patent illustrates an inflatable annulus augmentation device which includes a door-like flap. The device is placed inside the disc to hold material when the flap is closed and secured.

A similar device is described in U.S. Pat. No. 5,888,220 herein incorporated by reference. The device includes a deflated balloon which is inserted into the nucleus and then releases a mass of curable biomaterials. As shown in FIG. 1 of the '220 patent; the invention includes a delivery cannula and a balloon. The balloon is capable of being positioned within an intervertebral disc space and there filled with biomaterial delivered through the cannula.

It will be apparent to one of skill in the art that the subject invention may be used with any disc augmentation device known in the art including those described above.

Nucleus Decompression System

The spinal access devices may also be used in systems for treating disc degeneration that include nucleus decompression devices. A nucleus decompression device removes the disc nucleus tissue either by dissection, suction, dissolving, or by shrinking the nucleus. Various types of thermal energy are known to shrink the nucleus such as resistive heat, radiofrequency, coherent and incoherent light, microwave, ultrasound or liquid thermal jet energies. Decompression of the disc nucleus results in the protruded disc material collapsing toward the center of the disc thereby reducing the pressure on the spine nerve roots thereby minimizing or reducing the associated pain, weakness and/or numbness in the lower extremities, upper extremities, or neck region.

The subject spinal access device may be used for accessing the nucleus and delivering a nucleus decompression device. For example, a decompression device may be advanced from one of the working channels in the spinal access device.

The combination of the subject spinal access device with a decompression device results in increased tactile sensation for the surgeon thereby allowing the surgeon to atraumatically manipulate surrounding tissue to accurately deliver the decompression device. The decompression device may be any of a wide variety of devices suited for decompressing the nucleus. By utilizing the subject spinal access device, nucleus decompression devices well-known in the art may be improved as a result of the real time, on-board visualization capabilities and the creation of a working area.

As illustrated in FIG. 34, the subject spinal access device 110 may be used to deliver a decompression device instead of an augmentation device. The decompression device may be detachably disposed on a distal end 130 of a catheter 135. The device may be directly introduced or steered into position using the direct visualization capabilities of the subject devices similar to the delivery of augmentation devices described above. For example, the nuclear decompression devices would be advanced through a working channel 114 of the subject spinal access device 110. The spinal access device 110 includes a pair of working channels 113 and 114 in a distal end 112. The device may also include an aspiration port and an irrigation port as well. The visualization port is located within the spinal access device and covered by a shaped atraumatic tip 118. The visualization port is used by illumination, visualization and/or imaging components to provide direct visualization capabilities for implanting the disc augmentation device. In this particular embodiment, the spinal access device 110 is illustrated within a conventional trocar or introducer 102. The nuclear decompression device would be advanced through the working channel 114 and out onto the distal tip of the steerable catheter and positioned into the working area created by the atraumatic manipulation device 120.

In one aspect of the invention, the manipulation device remains in place while the decompression device is in use. In another aspect, the manipulation device may remain in a deployed state and detached from the catheter. In this way, the manipulation device remains in place to provide working access while also freeing the working channel and the catheter for other tasks related to the decompression of the nucleus.

In an exemplary method to access the nucleus, a trocar is first introduced using a conventional percutaneous approach. The subject steerable spinal access device is advanced through the trocar lumen and into the epidural space. The atraumatic tip may further move the epidural fat and other tissue in the epidural space to aid in the advancement of the steerable spinal device towards the disc nucleus. The dura and surrounding tissue are further moved by the deployment action of the atraumatic manipulation device to create a work space adjacent to the nucleus. In addition, the creation of a working space enhances the visualization capabilities by clearing a viewing area. Furthermore, visualization may further be improved by the introduction of saline or a cleaning solution into the working area. Finally, the disc decompression device is attached to a catheter and delivered to the nucleus or proximate to the nucleus via the working channel.

The decompression device may be any decompression device known in the art.

The following devices are exemplary of the decompression devices which may be used with the subject devices and in no way should be construed as limitations.

The subject device may deliver the decompression device described in US. Application No. 2002/0138091, herein incorporated by reference. As illustrated in FIGS. 1 and 2 of the application, the decompression device includes a handpiece and a connected tissue removal mechanism. The tissue removal mechanism is generally structured to draw tissue into the cannula by a pumping action produced by rotation of the rotatable element. As such, the rotational element and the cannula are structured to cooperatively engage to form or create a source of suction effective in drawing the nucleus pulposis into the cannula in response to the rotation of the rotational element.

U.S. Pat. No. 5,285,795 which is herein incorporated by reference provides another example of a decompression device which may be used with the subject spinal access device. The device includes a bendable probe that may bend more than 90° and still function in removing tissue. As shown in FIGS. 1 and 2 of the '795 patent, the bendable probe includes an elongate tubular cutting member which has at its end a flared guillotine-type cutter in order to allow fluid irrigation. Severed tissue and irrigation fluid is then aspirated through the tubular cutting member.

The subject spinal access device may also deliver the decompression device disclosed in U.S. Pat. No. 5,383,884, herein incorporated by reference. The decompression device includes a cutter secured to the end of a drive shaft and positioned so that each rotation of the cutter shaves off a segment of the nucleus. FIG. 2 of the '884 patent depicts the cutter in greater detail. The end is slotted on one side to provide a cutting window for progressively shaving away the herniated disc. The auger-like profile of the cutter transports the shaved segments backward to the annulus. The shavings are then aspirated to a collection vessel connected to an evacuation port on the device.

Additionally, the decompression device may use a temperature-controlled energy element to shrink the nucleus pulposis. The energy element removes some water and permits the nucleus pulposus to withdraw. For example, the decompression device may provide a high energy laser beam similar to the device described in U.S. Pat. No. 5,084,043 herein incorporated by reference. The laser beam vaporizes part of the nucleus instead of removing it mechanically.

The nucleus decompression device may also include a channeling mechanism like the device described in U.S. Pat. No. 6,264,650, the disclosure of which is incorporated by reference. The '650 device forms small holes within the disc and then applies thermal energy. FIG. 1 of the '650 patent illustrates the handpiece of the device, which includes an array of electrode terminals at its distal end. The handpiece connects to a power supply for providing high frequency voltage to a target site in order to reduce the volume of the nucleus thereby relieving pressure on the surrounding nerves.

Another example of a decompression device which may be delivered by the above spinal access device is disclosed in U.S. Application No. 2005/0149011. The device includes a catheter in which a probe is located at its distal end. FIGS. 2A and 2B of the application provides an exemplary embodiment of the catheter inserted into the lumen of an introducer. The catheter includes a handle stem and a probe section in which functional elements for the delivery of energy may be placed. The probe section delivers energy to the nucleus in order to shrink the nucleus pulposus.

It will be apparent to one of skill in the art that the subject invention may be used with any nucleus decompression device well known in the art including those described above.

Delivery of Pharmacological Agents and Other Compounds

Embodiments of the spinal access device of the present invention may also be used to more precisely inject, place, apply, dispense or otherwise administer pharmacological agents or other compounds directly into the spinal space. Advantageously, the direct visualization feature of embodiments of the spinal access device allow for more precise administration of pharmacological agents than conventional techniques. For example, the spinal access device could be positioned as described herein and the injection location visually confirmed using direct visualization. Thereafter, one of the working channels of the device may be used to introduce a needle or applicator to dispense pharmacological agents to the desired and visually confirmed location. In one aspect, the pharmacological agent includes an active ingredient that is a drug to treat and/or prevent a disorder of the spine. Examples of an active agent include: an anti-inflammatory agent, an analgesic agent, an anesthetic agent, an anti-cicatrizant agent, a wound healing agent or a lysis inducing agent and combinations thereof. Another specific example includes the use of the spinal therapy device to administer one or more injections into the spinal space such as in administering a nerve block. In another specific embodiment, the spinal access device could be used to perform wound therapies. The precise access provide by the access tools described herein could be used to deliver of a number of wound treatments including, for example, the delivery and use of a wide variety of dressings including alginates, hydrocolloids, transparent films, foams, amorphous hydrogels and hydrogel sheet wound covers. Additionally, the working channels of the spinal access device may be utilized to perform debrider procedures including mechanical and enzymatic debrider techniques. In addition, the spinal access device may be used as a platform to perform tissue or cell therapy, dispense cultivated disc cells, spinal tissue cells, synthetic or tissue engineered polymers or other compounds to perform spine based therapies. It is to be appreciated that the direct visualization capabilities of embodiments of the spinal access device of the present invention bring new precision and certainty to these and other procedures.

Diagnostic Methods

It is contemplated that the subject spinal access device may also be used for diagnostic purposes. Because of the complexity of the spine, it is often more difficult to diagnose an injury than for other medical conditions. As such, the direct visualization capabilities of the subject devices may be able to accurately identify any instability or deformity in the spine. For example, the subject device offers direct visualization of any tumors, fractures, nerve damage, or disc degeneration. In addition, the subject devices may include sensors for collecting diagnostic data, for example, sensors that measure flow, pressure, or oxygen concentration. The subject devices may also be used to remove fluid, tissue or bone samples to be used for external diagnostic tests. Additionally, the subject devices may deliver testing reagents or additional instruments for diagnosing disc degeneration and bony degeneration, for example, the subject devices may deliver electrodes for diagnosis and treatment.

Kits

Also provided by the subject invention are kits for use in practicing the subject methods. The kits of the subject invention include at least one spinal access device. The kits may include an expanding element such as mesh, a balloon or an expanding atraumatic element. The kits may also include at least one visualization device as well as a material or marker to enhance visualization of the structure using an imaging modality outside of the body. Furthermore, the kits may include any of the additional devices discussed above, such as a disc augmentation device or nucleus decompression device. The kits may also include an active agent to treat and/or prevent a disorder of the spine such as an anti-inflammatory agent, an analgesic agent, an anesthetic agent, an anti-cicatrizant agent, a wound healing agent or a lysis inducing agent. Additionally, the kits may include at least one sensor for collecting diagnostic data. Finally, the kits may further include instructions for using the subject devices for diagnosing and treating disc degeneration and bony degeneration.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While embodiments of the present invention have been shown and described herein, it will be obvious to those skilled in the art that such embodiments are provided by way of example only. Numerous variations, changes, and substitutions will now occur to those skilled in the art without departing from the invention. For example, the relative sizes of the device delivery and shield delivery catheters may vary with specific applications and spinal therapies whereby the device delivery catheter may be larger than the shield delivery catheter and vice versa. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating intervertebral disc degeneration, comprising:
introducing a spinal access device having direct visualization capability into a portion of the spine;
steering the spinal acess device to a position adjacent an outer surface of the spinal dura matter using visualization information provided by the spinal access device;
displacing the spinal dura matter with a portion of the spinal access device to create a working area; and
using the spinal access device to deliver a disc augmentation device for treating intervertebral disc degeneration.

2. The method according to claim 1 wherein the augmentation device provides structural support to the annulus.

3. The method according to claim 1 wherein the augmentation device seals a torn annulus.

4. The method according to claim 1 wherein the augmentation device adds additional material to the nucleus.

5. A method for treating intervertebral disc degeneration, comprising:
introducing a spinal access device having direct visualization capability into a portion of the spine;
steering the spinal acess device to a position adjacent an outer surface of the spinal dura matter using visualization information provided by the spinal access device;
displacing the spinal dura matter with a portion of the spinal access device to create a working area; and
using the spinal access device to deliver a nucleus decompression device for treating intervertebral disc degeneration.

6. The method according to claim 5 wherein the nucleus decompression device removes a portion of the nucleus.

7. The method according to claim 5 wherein the nucleus decompression device shrinks a portion of the nucleus.

8. A system for intervertebral disc augmentation comprising:
a spinal access device configured to deliver a disc augmentation device to an intervertebral disc comprising:
at least one disc augmentation device;
an elongate body;
a direct visualization device; and
a working channel.

9. The system of claim 8 wherein augmentation includes repairing a herniated disc.

10. The system of claim 8 wherein augmentation includes supporting a damaged annulus.

11. The system of claim 8 wherein augmentation includes sealing an annulus.

12. The system of claim 8 wherein augmentation includes the addition of material to the nucleus.

13. The system of claim 8 wherein the spinal access device includes an expanding structure.

14. The system of claim 13 wherein the expanding structure is mesh, a balloon, or an atraumatic element.

15. The system of claim 13 wherein expanding the expanding structure deforms a portion of the spinal dura matter.

16. The system of claim 13 wherein expanding the structure creates a working area.

17. The system of claim 8 wherein the visualization information is provided from an image generated by a sensor located on the instrument.

18. The system of claim 8 wherein the augmentation device further comprises at least one mesh, cage, barrier, patch, scaffold, sealing means, hydrogels, silicones, or growth factors.

19. The system of claim 8 wherein the augmentation device is an ablation device.

20. A system for intervertebral nuclear decompression comprising:
a spinal access device configured to deliver a nuclear decompression device to an intervertebral disc comprising:
a nuclear decompression device;
an elongate body;
a direct visualization device;
a working channel; and
a dissection tip.

21. The system according to claim 20 wherein the decompression device further comprises a temperature-controlled energy element.

22. The system according to claim 21 wherein the energy element may be a thermal energy device that delivers resistive heat, radiofrequency, coherent and incoherent light, microwave, ultrasound or liquid thermal jet energies to the nucleus.

23. The system of claim 20 wherein the spinal access device includes an expanding structure.

24. The system of claim 23 wherein the expanding structure is mesh, a balloon, or an atraumatic element.

25. The system of claim 23 wherein expanding the expanding structure deforms a portion of the spinal dura matter.

26. The system of claim 23 wherein expanding the structure creates a working area.

27. The system of claim 20 wherein the visualization information is provided from an image generated by a sensor located on the instrument.

28. A method of diagnosing disc degeneration in a patient, said method comprising:
introducing a spinal access device having direct visualization capability into a portion of the spine;
steering the spinal access device using visualization information provided by the spinal access device;
displacing the spinal dura matter with a portion of the spinal access device to create a working area; and
assessing the condition of one or more intervertebral discs.

29. The method according to claim 28 wherein the spinal access device comprises a material or marker to enhance visualization of the structure using an imaging modality outside of the body.

30. The method according to claim 29 further comprising receiving visualization information from an imaging modality outside of the body.

31. The method according to claim 30 wherein the imaging modality comprises fluoroscopy.

32. The method according to claim 30 wherein the imaging modality comprises magnetic resonance imaging.

33. The method according to claim 28 wherein the visualization information is provided from an image generated by a sensor located on the instrument.

34. The method according to claim 28 wherein the spinal access device includes a sensor for collecting diagnostic data.

35. A kit for augmenting the intervertebral disc, the kit comprising:
at least one disc augmentation device;
a spinal access device having direct visualization capabilities; and
instructions for implanting the at least one disc augmentation device using the spinal access device.

36. A kit for decompressing the nucleus of an intervertebral disc, the kit comprising:
at least one nucleus decompression device;
a spinal access device having direct visualization capabilities; and
instructions for decompressing the nucleus of an intervertebral disc using the spinal access device.

37. A method for treating intervertebral disc degeneration, comprising:
introducing a spinal access device having direct visualization capability into a portion of the spine;
displacing the spinal column matter with a portion of the spinal access device to create a working area; and
using the spinal access device to deliver a disc augmentation device for treating intervertebral disc degeneration.

38. The method according to claim 37 wherein the spinal access device is steered to a position within the spinal column using the direct visualization capability of the spinal access device.

39. The method according to claim 37 wherein the augmentation device provides structural support to the annulus.

40. The method according to claim 37 wherein the augmentation device seals a torn annulus.

41. The method according to claim 37 wherein the augmentation device adds additional material to the nucleus.

42. A method for treating intervertebral disc degeneration, comprising:
introducing a spinal access device having direct visualization capability into a portion of the spine;
displacing the spinal column matter with a portion of the spinal access device to create a working area; and
using the spinal access device to deliver a nucleus decompression device for treating intervertebral disc degeneration.

43. The method according to claim 42 wherein the spinal access device is steered to a position within the spinal column using the direct visualization capability of the spinal access device.

44. The method according to claim 42 wherein the nucleus decompression device removes a portion of the nucleus.

45. The method according to claim 42 wherein the nucleus decompression device shrinks a portion of the nucleus.

46. A method for treating intervertebral disc degeneration, comprising:
introducing a spinal access device having direct visualization capability into a portion of the spine;
displacing the spinal column matter with a portion of the spinal access device to create a working area; and
using the spinal access device to deliver a stimulation electrode device for treating intervertebral disc degeneration.

47. The method according to claim 46 wherein the spinal access device is steered to a position within the spinal column using the direct visualization capability of the spinal access device.

48. A method for treating intervertebral disc degeneration, comprising:
introducing a spinal access device having direct visualization capability into a portion of the spine;
steering the spinal access device using visualization information provided by the spinal access device;
displacing the spinal column matter with a portion of the spinal access device to create a working area; and
using the spinal access device to deliver a stimulation electrode device for treating intervertebral disc degeneration.

49. A spinal access device configured to deliver a disc augmentation device to an intervertebral disc, said device comprising:
a therapeutic device;
an elongate body;
a direct visualization device; and
at least one working channel.

50. A system for intervertebral nuclear decompression comprising:
a spinal access device configured to deliver a nuclear decompression device to an intervertebral disc comprising:
a nuclear decompression device;
an elongate body;
a direct visualization device; and
a working channel.

51. A method of diagnosing disc degeneration in a patient, said method comprising:
introducing a spinal access device having direct visualization capability into a portion of the spine;
displacing the spinal dura matter with a portion of the spinal access device to create a working area; and
assessing the condition of one or more intervertebral discs.

52. A system for treating spinal disease comprising:
a spinal access device configured to deliver a therapeutic device to a spinal column comprising:
a therapeutic device;
an elongate body;
a direct visualization device; and
at least one working channel.

53. A system for treating spinal disease comprising:
a spinal access device configured to deliver a therapeutic device to a spinal column comprising:
a therapeutic device;
an elongate body;
a direct visualization device;
at least one working channel; and
at least one irrigation or aspiration channel.
